(19) United States
(12) Patent Application Publication
(10) Pub. No.: US 2026/0060623 A1
(43) Pub. Date: Mar. 5, 2026

(54) RADIATION SHIELDING APPARATUS, METHOD OF MAKING AND USING THE SAME

(71) Applicant: Salus Scientific Corp., Charleston, SC (US)

(72) Inventor: Nicholas Randolph Tomczak, Richlands, NC (US)

(21) Appl. No.: 19/313,592

(22) Filed: Aug. 28, 2025

Related U.S. Application Data
(60) Provisional application No. 63/688,144, filed on Aug. 28, 2024.
(60) Provisional application No. 63/863,304, filed on Aug. 13, 2025.

(51) Int. Cl. A61B 6/10; A61B 6/00; A61B 6/04; G21F 1/12; G21F 3/00

(52) U.S. Cl. CPC A61B 6/107; A61B 6/0442; A61B 6/54; A61B 6/56; G21F 1/12; G21F 3/00

(57) ABSTRACT

A system configured to reduce scatter radiation around a surgical table during a medical procedure that may include at least one radiation shield configured to be connected to a surgical table with an attachment mechanism, wherein the at least one radiation shield includes one or more adjustable radiation shield segments coupled or releasably coupled to the at least one radiation shield. A device may include a pump in fluid communication with the one or more adjustable radiation shield segments comprising a pneumatic channel that is configured to at least partially inflate the pneumatic channel such that at least a first portion of the at least one radiation shield extends vertically above at least a portion of the surgical table, wherein a second portion of radiation shield is configured to extend below the surgical table. A device may include a controller configured to activate the pump.

### CROSS REFERENCE TO RELATED APPLICATIONS

This application claims the benefit of U.S. Provisional Patent Application No. 63/688,144, filed Aug. 28, 2024, and U.S. Provisional Patent Application No. 63/863,304, filed Aug. 13, 2025, the entire contents of each of the foregoing applications are hereby incorporated by refence as if fully set forth herein.

### FIELD OF THE INVENTION

The present invention, relates to radiation shielding system, apparatus, method of using the same and more particularly, to an adjustable radiation shielding configured to protect medical personnel from exposure to radiation, e.g., scatter radiation, during medical procedures.

### BACKGROUND OF THE INVENTION

The problem involves inadequate radiation shielding during medical procedures. For example, the use of C-arm fluoroscopy in medical procedures has become an essential tool for guiding interventions, but it poses significant radiation risks to both patients and healthcare providers. The real-time imaging capability relies on ionizing radiation, and prolonged or repeated exposures can increase cumulative dose. For patients, this may result in skin injuries, hair loss, or, in rare cases, radiation-induced cancer when high doses are involved. Because many procedures require extended fluoroscopy time, careful monitoring and dose management are critical to reduce unnecessary exposure.

A related concern is scatter radiation, which occurs when X-rays deflect off various items including but not limited to the people, medical equipment, and spread into the surrounding environment. Physicians, nurses, and technologists who remain close to the C-arm during procedures are repeatedly exposed to this secondary radiation. Over time, such exposure can increase the risk of cataracts, thyroid disorders, car damage, and certain cancers, particularly if protective equipment is not consistently used. Even with conventional equipment such as lead aprons, thyroid shields, and lead glasses, there is inadequate shielding that may leave physicians, nurses, and technologists who remain vulnerable to harmful doses of radiation overtime. Also, beyond the primary beam and scatter radiation, other radiation hazards may include leakage from the C-arm tube housing and cumulative exposure from performing multiple procedures in a single day. These exposures may not cause immediate harm but can contribute to long-term health issues for physicians, nurses, and technologists.

There is a need for an apparatus, system and/or method that protects or mitigates medical physicians, nurses, and technologists who from exposure to radiation during medical procedures.

### SUMMARY OF THE INVENTION

Accordingly, the invention is directed to a radiation shielding apparatus, method of making and using the same configured to reduce scatter radiation around a surgical table during a medical procedure

An advantage of the invention is to provide a system that allows for extension of one or more radiation shields attached to surgical table with a control unit.

Yet another advantage of the invention is to provide a system for retraction of one or more radiation shields attached to surgical table with a control unit.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, a system is configured to reduce scatter radiation around a surgical table during a medical procedure. The system includes at least one radiation shield configured to be connected to a surgical table with an attachment mechanism. The at least one radiation shield includes one or more adjustable radiation shield segments coupled or releasably coupled to the at least one radiation shield. The system also includes a pump in fluid communication with the one or more adjustable radiation shield segments. The one or more adjustable radiation shield segments include a pneumatic channel that is configured to at least partially inflate the pneumatic channel such that at least a first portion of the at least one radiation shield extends vertically above at least a portion of the surgical table, a second portion of radiation shield is configured to extend below the surgical table. A controller configured to activate the pump.

In another aspect of the present invention, a system is configured to reduce scatter radiation around a surgical table during a medical procedure. The system includes at least one flexible radiation shield comprising a first end, a second end spaced apart from the first end, a first side and a second side spaced apart from the first side, the at least one flexible radiation shield is configured to be attached to a surgical table with an attachment mechanism. The at least one flexible radiation shield includes one or more adjustable radiation shield segments including one or more air channels. The system further has a pump in fluid communication with the one or more air channels. The pump is configured to at least partially inflate the one or more air channels such that at least a first end of the at least one flexible radiation shield extends vertically above at least a portion of the surgical table, and a second end of flexible radiation shield is configured to extend below the surgical table. The system includes a controller in communication with a control unit, wherein the control unit is configured to activate the pump.

In yet another aspect of the present invention, a system is configured to reduce scatter radiation around a surgical table during a medical procedure. The system includes at least one flexible radiation shield including a first end, a second end spaced apart from the first end, a first side and a second side spaced apart from the first side where the at least one flexible radiation shield is configured to be attached to a surgical table with an attachment mechanism. The at least one flexible radiation shield includes one or more adjustable radiation shield segments including one or more air channels and one or more stiffing members. They system includes a pump in fluid communication with the one or more air channels where the pump is configured to at least partially inflate the one or more air channels such that at least a first end of the at least one flexible radiation shield extends vertically above at least a portion of the surgical table and a second end of flexible radiation shield is configured to extend below the surgical table. The system also includes a controller in communication with a control unit, wherein the control unit is configured to activate the pump.

In still yet another aspect of the present invention, a system includes a safety apparatus configured to reduce scatter radiation around a surgical table during a medical procedure. The system includes at least one flexible radiation shield including a first end, a second end spaced apart from the first end, a first side and a second side spaced apart from the first side, the at least one flexible radiation shield is configured to be attached to a surgical table with an attachment mechanism. The at least one flexible radiation shield includes one or more adjustable radiation shield segments where the at least one flexible radiation shield comprises a material having a lead equivalence (LE) in a range from about 0.125 mm to about 1.0 mm or greater.

In still yet another aspect of the present invention, a system includes an apparatus configured to extend a portion of a surgical table. The apparatus includes a generally planar body having a top surface and a bottom surface and a recess in the generally planar body. The recess being open on one side and bounded on three sides by edge portions of the generally planar body including the top surface, the bottom surface and side surfaces extending from the top surface and the bottom surface, the recess is configured to receive at least a portion of the surgical table. The apparatus includes a material that is substantially transparent to x-ray radiation.

In still yet another aspect of the present invention, an apparatus is configured to extend a portion of a surgical table. The apparatus includes a generally planar body having a top surface and a bottom surface configure to be attached mechanism to a portion the surgical table in order to increase a circumference of the surgical table.

In still yet another aspect of the present invention, a method of operating the apparatus according includes providing at least one radiation shield configured to be connected to a surgical table with an attachment mechanism. The at least one radiation shield includes one or more adjustable radiation shield segments coupled or releasably coupled to the at least one radiation shield, a pump in fluid communication with the one or more adjustable radiation shield segments including a pneumatic channel configured to at least partially inflate the pneumatic channel such that at least a first portion of the at least one radiation shield extends vertically above at least a portion of the surgical table and where a second portion of radiation shield is configured to extend below the surgical table; and a controller configured to activate the pump. The method includes attaching the at least one radiation shield to the surgical table and activating the pump to partially inflate the pneumatic channel and raise at least the first portion of the at least one radiation shield vertically above a portion of the surgical table.

This Summary section is neither intended to be, nor should be, construed as being representative of the full extent and scope of the present disclosure. Additional benefits, features and embodiments of the present disclosure are set forth in the attached figures and in the description hereinbelow, and as described by the claims. Accordingly, it should be understood that this Summary section may not contain all of the aspects and embodiments claimed herein.

Additionally, the disclosure herein is not meant to be limiting or restrictive in any manner. Moreover, the present disclosure is intended to provide an understanding to those of ordinary skill in the art of one or more representative embodiments supporting the claims. Thus, it is important that the claims be regarded as having a scope including constructions of various features of the present disclosure insofar as they do not depart from the scope of the methods and apparatuses consistent with the present disclosure (including the originally filed claims). Moreover, the present disclosure is intended to encompass and include obvious improvements and modifications of the present disclosure.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 illustrates an exemplary perspective view of a radiation shielding system arranged on a surgical table in an extended orientation according to an embodiment of the invention.

FIG. 2 illustrates an exemplary left side view of FIG. 1 according to an embodiment of the invention.

FIG. 3 illustrates an exemplary right side view of FIG. 1 according to an embodiment of the invention.

FIG. 4 illustrates a exemplary top view of FIG. 1 according to an embodiment of the invention.

FIG. 5 illustrates an exemplary partial perspective front view of a flexible radiation shield of FIG. 1 in a non-extended orientation with a manifold according to an embodiment of the invention.

FIG. 6 illustrates an exemplary rear perspective front view of FIG. 5 in a non-extended orientation with a manifold according to an embodiment of the invention.

FIG. 7 illustrates an exemplary rear perspective front view of FIG. 5 in a non-extended orientation without a manifold according to an embodiment of the invention.

FIG. 8 illustrates an exemplary cross-sectional view of a FIG. 7 along line 525 to 526.

FIG. 9 illustrates an exemplary perspective view of a manifold of FIG. 5 according to an embodiment of the invention.

FIG. 10 illustrates an exemplary perspective front view of a system including a flexible radiation shield in a non-extended orientation according to an embodiment of the invention.

FIG. 11 illustrates a partial top view of FIG. 10 according to an embodiment of the invention.

FIG. 12 illustrates a perspective side view of FIG. 10 according to an embodiment of the invention.

FIG. 13 illustrates a perspective inner view of the flexible radiation shield of FIG. 10 without pneumatic channel port covers according to an embodiment of the invention.

FIG. 14 illustrates a perspective inner view of the flexible radiation shield of FIG. 10 with pneumatic channel port covers in an uninstalled configuration according to an embodiment of the invention.

FIG. 15 illustrates a perspective inner view of the flexible radiation shield of FIG. 10 with pneumatic channel port covers installed according to an embodiment of the invention.

FIG. 16 illustrates a perspective side otter view of the flexible radiation shield of FIG. 10 in an expanded configuration according to an embodiment of the invention.

FIG. 17 illustrates a perspective view of the flexible radiation shield of FIG. 10 according to an embodiment of the invention.

FIG. 18 illustrates a perspective view of FIG. 10 with the adjustable radiation shield segments in a partially detached configuration according to an embodiment of the invention.

FIG. 19 illustrates a diagram of a radiation shielding system according to an embodiment of the invention.

FIG. 20 illustrates a perspective side view of a table extension for a surgical table according to an embodiment of the invention.

FIG. 21 illustrates a top view of FIG. 20 according to an embodiment of the invention.

FIG. 22 illustrates a perspective rear end view of a table extension for a surgical table according to an embodiment of the invention.

FIG. 23 illustrates a perspective rear end view of the table extension of FIG. 22 according to an embodiment of the invention.

FIG. 24 illustrates a cross-section view of the table extension of FIG. 22 along line 2201 according to an embodiment of the invention.

FIG. 25 illustrates a perspective side view of the table extension of FIG. 22 in an installed configuration according to an embodiment of the invention.

FIG. 26 illustrates a top view of the table extension of FIG. 22 in an installed configuration according to an embodiment of the invention.

FIG. 27 illustrates a bottom side inside perspective view of the table extension of FIG. 22 with a flexible radiation shield installed configuration according to an embodiment of the invention.

FIG. 28 illustrates a side perspective view of the table extension of FIG. 22 with a flexible radiation shield installed configuration according to an embodiment of the invention.

FIG. 29 illustrates a top perspective view of the table extension of FIG. 22 with an extension unit in a first orientation according to an embodiment of the invention.

FIG. 30 illustrates a top perspective view of the table extension of FIG. 22 with an extension unit in a second orientation according to an embodiment of the invention.

FIG. 31 illustrates a top view of the table extension of FIG. 22 with an extension unit and a bracket in a second orientation according to an embodiment of the invention.

FIG. 32 illustrates a bottom view of the table extension, extension unit and bracket of FIG. 31.

FIG. 33 illustrates a side perspective of the table extension, extension unit and bracket of FIG. 31.

FIG. 34 illustrates a perspective right side view of a bracket according to an embodiment of the invention.

FIG. 35 illustrates a top view of the bracket of FIG. 34 according to an embodiment of the invention.

FIG. 36 illustrates a left side view of the bracket of FIG. 34 according to an embodiment of the invention.

FIG. 37 illustrates a bottom view of the bracket of FIG. 34 according to an embodiment of the invention.

FIG. 38 illustrates a right side view of the bracket of FIG. 34 according to an embodiment of the invention.

FIG. 39 illustrates a left side view of the bracket of FIG. 34 with a table apparatus according to an embodiment of the invention.

FIG. 40 illustrates a partial perspective view of a bracket of FIG. 34 coupled to a surgical table and attachment table.

FIG. 41 illustrates a partial perspective view of a bracket of FIG. 34 coupled to a surgical table and attachment table.

FIG. 42 illustrates a method of utilizing the extension table for the surgical table.

### DETAILED DESCRIPTION OF THE ILLUSTRATED EMBODIMENTS

For the purposes of promoting an understanding of the principles of the present disclosure, reference will now be made to the exemplary embodiments illustrated in the drawing(s), and specific language will be used to describe the same.

Appearances of the phrases an “embodiment,” an “example,” or similar language in this specification may, but do not necessarily, refer to the same embodiment, to different embodiments, or to one or more of the figures. The features, functions, and the like described herein are considered to be able to be combined in whole or in part one with another as the claims and/or art may direct, either directly or indirectly, implicitly or explicitly.

Any singular reference to an element, component, or step herein should be understood as including one or more of such elements, components, or steps unless the context clearly dictates otherwise. Similarly, references to “a,” “an,” or “the” invention are intended to encompass both singular and plural forms of the disclosed subject matter.

As used herein, “comprising,” “including,” “containing,” “is,” “are,” “characterized by,” and grammatical equivalents thereof are inclusive or open-ended terms that do not exclude additional unrecited elements or method steps unless explicitly stated otherwise.

In order to more fully appreciate the present disclosure and to provide additional related features, each of the following references are incorporated therein by reference in their entirety and for the teachings described as follows:
(1) U.S. Pat. Application Publication No. 10,709,395, by Stegehuis, et al., which discloses a radiation system including a patient support platform. An X-ray radiation source is positioned beneath the patient support platform and enclosed by fixed radiation shielding. An X-ray radiation detector is positioned above the patient support platform. A detector X-ray radiation shield comprising shield extension is arranged on either side of the patient support platform, which extend from the X-ray radiation 5 detector to the fixed radiation shielding. The shield extensions are able to be moved relative to the source radiation shield to allow access to a patient on the support platform.
(2) U.S. Pat. No. 11,931,304, by Wilson, which discloses a mattress system is provided that is optimized for the hospital setting and includes a guiderail system that accepts a variety of accessories for attachment thereto. The guiderail system may have integrated data lines, power lines, gas lines, and/or fluid lines. Also provided are radioabsorbant shields, trays and other components designed for optimal use with the mattress system.
(3) U.S. Pat. No. 11,937,957, by YiFat, which discloses apparatuses (devices, systems) and methods for shielding (protecting) surroundings around periphery of regions of interest located inside objects (e.g., patients) from radiation emitted by X-ray systems towards the objects. Apparatus includes: at least one radiation shield assembly including a support base connectable to an X-ray system radiation source or detector, and a plurality of radiation shield segments sequentially positioned relative to the support base, thereby forming a contiguous radiopaque screen configured for spanning around the region of interest periphery with a radiopaque screen edge opposing the object. Radiation shield segments are individually, actively controllable to extend or contract to selected lengths with respective free ends in directions away from or towards the support base(s), for locally changing contour of the radiopaque screen edge. Applicable for shielding (protecting) medical personnel, and patients, from exposure to X-ray radiation during medical interventions or/and diagnostics.
(4) U.S. Pat. Application Publication No. 2014/0048730, by Niedzielski, et al., which discloses a radiation protection system for protecting medical personnel from radiation being applied from a radiation source to a patient positioned on a table that includes a radiation-shielding wall including, upper shield suspended by a gas spring lift arm, the upper shield consisting of translucent radiation resistance window, with a left and right side of flexible radiation shielding material, that telescopes down on each side of the table to form a complete radiation barrier. The shield is positioned above the table. The shield also has a radiation-shielding flexible interface attached to the radiation shielding window that covers a portion of the patient.
(5) U.S. Pat. Application Publication No. 2023/0126167, by Wilson, et al., which discloses a flexible radiation shielding system for reducing scatter radiation that may arise during the performance of certain medical imaging procedures. A multi-articulated shielding system comprising two or more shielding elements hingedly coupled to each other to thereby enable a user to bend the shielding system into a desired shape to provide radiation shielding protection to workers. A flexible radiation shielding system may comprise a plurality of shielding elements that are, for example, translucent, transparent, clear, etc., to enable workers to view objects through the shielding elements.
(6) U.S. Pat. Application Publication No. 20240079156, by YiFat, which discloses rigid structures and composite materials thereof for providing radiation attenuation/shielding. Some embodiments pertain to a radiation shielding apparatus including: a plurality of positionable radiation-shielding stacks of tiles. The stacks are subsequently and adjacently arranged in a contiguous configuration. A tile positioning mechanism allows movement of tiles within a stack between a stacked (retracted) position and an extended position. In the extended position, the tiles of each of the plurality of radiation shielding stacks at least partially overlap tiles of subsequent and adjacent tile stack at corresponding opposing side-margins thereof.
(7) U.S. Pat. Application Publication No. 20240293093 by Ubel, et al., which discloses radiation shielding devices that shield radiation from multiple directions are described. In one embodiment, a radiation shielding device is provided, including a first shielding portion, a second shielding portion, and an attachment member configured to support the first shielding portion in a position relative to the second shielding portion. One or more features of the radiation shielding device provide structural support to at least a portion of the radiation shielding device, and/or maintain the radiation shielding device in the selected configuration.
(8) U.S. Pat. Application Publication No. 2025/0082963 by Shin et al, which discloses a medical radiation shielding device including a bed irradiated with radiation from an outside, a shielding cover that is provided to be movable in a longitudinal direction of the bed and forms a radiation shielding space for shielding the radiation in the bed, a shielding seat that is provided to be movable in the longitudinal direction of the bed, is disposed between the bed and the shielding cover, and shields the radiation radiated to the radiation shielding space, and a pair of holders to which the shielding cover and the shielding seat are independently coupled to be movable in the longitudinal direction of the bed.
(9) U.S. Pat. Application Publication No. 2025/0127466 by Wilson, et al., which discloses A shielding system for reducing scatter radiation during x-ray imaging procedures may include a drawer feature to temporarily move a shield portion laterally outward from a medical procedure table. A shielding system for reducing scatter radiation during x-ray imaging procedures may include a curtain feature to temporarily move a shield portion to enable a range of positioning of an x-ray source around a medical procedure table; in some embodiments, the shield portion may include wound wire stays to flexibly and/or temporarily conform to movement of equipment near the medical procedure table. A shielding system for reducing scatter radiation during x-ray imaging procedures may include a rail extension feature to enable mounting or flexible positioning of components near a medical procedure table; in some aspects, the rail extension may include an undercut region that facilitates placement of jam boards and the like under a patient during a procedure. A shielding system for reducing scatter radiation during x-ray imaging procedures may include a telescoping hip shield feature configured to releasably couple to a rail of a medical procedure table and to extend and retract longitudinally to vary the positioning of the telescoping shield. A shielding system for reducing scatter radiation during x-ray imaging procedures may include a head protection feature configured to pivot a shield portion about two or more axes relative to a medical procedure table.
(10) U.S. Pat. Application Publication No. 2025/0195017 by Foster, et al., which discloses a radiation shield assembly is described, configured to block radiation emanating from a radiation source from reaching a user. Two shields are supported by a support arm, and are configured to rotate and translate relative to one another about the support arm's longitudinal axis. This allows the shield to be easily configured and reconfigured as necessary to visualize various parts of a patient's body via radiography. Sterile coverings are provided to ensure asepsis during a surgical procedure.

In some aspects as described herein, the system includes an adjustable and flexible radiation shielding system configured to protect medical personnel from exposure to radiation, e.g., scatter radiation, during medical procedures. During surgical procedures radiation is emitted from a radiation source, e.g., C-arm or another source used in the medical procedure. The use of C-arm fluoroscopy in medical procedures relies on radiation being emitting from under a surgical table at various angles. Scatter radiation occurs when x-rays from an emitter on the C-arm or other radiation source deflect off items including but not limited a surgical table, a patient and other medical hardware and spread into the surrounding environment. Medical personnel including but not limited physicians, nurses, and technologists who remain close to the C-arm or radiation source during procedures are repeatedly exposed to this scatter radiation and other radiation as described herein. The adjustable and flexible radiation shielding system is configured to configured to reduce their exposure to such radiation during the medical procedures.

In some aspects as described herein, the system includes an adjustable, flexible and soft radiation shielding system configured to protect medical personal while also easily permitting a patient to be easily arranged on and off the surgical table with minimal intervention from the medical personal.

In one embodiment, the radiation shielding system includes a plurality of soft and flexible radiation shields configured to be connected to a surgical table and arranged fully or partially around a circumference of the surgical table with an attachment mechanism. In this embodiment, there is no need for a pump, control unit or other activation mechanism as the flexible radiation shields are already configured in an extended configuration. By way of example, in this embodiment, the radiation shields can already be inflated and sealed in the extended configuration. Optionally and/or alternatively, the radiation shields are in a extended configuration by another mechanism, e.g., stiffing element, rigid material, e.g., plastic, thermoplastic, combinations of the same and the like or other.

The attachment mechanism can includes hook and loop fasting mechanism, snaps, magnets, combinations of the same. In a preferred embodiment, the attachment mechanism includes a hook and loop fasting mechanism, e.g., VELCRO.

In one embodiment, each of the flexible radiation shields can be individually controlled, controlled together or controlled in any predetermined scheme, e.g., one at time, two at time, three at time, etc., with a control unit where each of the flexible radiation shields are configured to raise and lower each above the surgical table to a predetermined height. In one embodiment, the control unit is in communication with controller configured to activate an extendable mechanism integral with each of the flexible radiation shields, coupled to each of the flexible radiation shields, and/or releasably coupled to each of the flexible radiation shields. The extendable mechanism can include one or more extendable elements operatable with an actuator, one or more pneumatic channels configured to be pressurized with a pressurization device, e.g., pump, materials configured to harden with electricity, and combinations of the same and the like.

In some aspects, an extendable mechanism is configured to extend and retract one or more of the flexible radiation shields above the surgical table. Each of the radiation shields includes one or flexible extendable radiation shield segments including one or more pneumatic channels, e.g., air channel or fluid channel. The pneumatic channels can have straight, curved or any angled geometry configured to extend the radiation shield to the desired location. In operation, the pneumatic channels are configured to at least partially inflate such that at least a portion of one or more of the flexible radiation shields extends vertically above at least a portion of the surgical table. Optionally and/or alternatively, depending on the length of the one or more of the flexible radiation shields a portion of radiation shield is extends below the surgical table.

In one embodiment, the flexible radiation shields can include a dual-pneumatic channel configuration for articulation of the shield assembly, allowing for improved maneuverability around an operating room environment during medical interventions.

In one embodiment, the flexible radiation shields are sized and positioned on the surgical table so they can extend vertically above the surgical table in a range from about three inches to 18 inches or greater. Optionally and/or alternatively, each of the flexible radiation shields are sized and positioned on the surgical table to extend below the surgical table to length of about three inches or more above the floor. Optionally and/or alternatively, each of the flexible radiation shields do not have to extend vertically or below the surgical table to the same height. For example, one flexible radiation shield can be sized to extend to a height lower than another flexible radiation shield. Of course, each of the flexible radiation shields can also be sized to extend below the surgical table at different heights above the ground. For example, one shield may be 2 inches to about 16 inches or greater.

In some aspects, the techniques described herein relate to a system, wherein pneumatic channel is configured to be inflated in a range from about 1 psi to about 50 psi or greater with the pump. In a preferred embodiment, the pressure of the pneumatic channel is in a range from about 3 psi to about 6 psi. The pneumatic channel can include one or more pneumatic channels and where can pneumatic channel can be integral with the flexible radiation shields, attached to the flexible radiation shields, or releasably attached to the flexible radiation shields.

In one embodiment, each of the pneumatic channels are in fluid communication with a pressurization source, e.g., pump, configured to pressurize the channels. Optionally and/or alternatively, the pump is connected a manifold or directly to each pneumatic channel. The channels are configured to be connected to an air pressured source, e.g., pump, that this controllable with a controller and control unit.

In one embodiment, the manifold is attached directly to the radiation shields such that more than channel of the pneumatic channels can be inflated ounce as described herein with one compressed air (or another gas) in communication with the manifold.

In one embodiment, the manifold, e.g., air manifold, is device including with valves used to distribute compressed air (or another gas) from a single source to multiple outputs with a controller and control unit.

In one embodiment, the control unit is configured to control the pneumatic channels individually or each channel corresponding to each flexible radiation shield. In one embodiment, the controller can be in a communication with a control unit. The control unit can configured to raise and lower each flexible radiation shield individually, one or more flexible radiation shield or a predetermined group of flexible radiation shield.

In one embodiment, the control unit is provided for individually actuating, extending, or retracting each radiation shield sections or segments relative to the surgical table, with the control unit including a touchscreen interface for user interaction. For example, the control unit can be configured to include zone-specific controls for individual radiation shield sections or segments, allowing for independent inflation or deflation of each section to a predetermined pressure level.

In one embodiment, the system is configured for rapid deflation capabilities by applying a negative pressure or vacuum one or more of the pneumatic channels, thereby enabling quick retraction of one or more of the radiation shield segments to permit emergency access to the patient. In some aspects, the techniques described herein relate to a system, wherein the pneumatic channel is configured to be deflated with a vacuum pressure in a range from about 10 3 to 10 9 Torr with the pump. However, it is noted, the system can also be retracted by releasing the pressure without the use of a negative pressure.

The control unit can controller can be in wireless communication over a network or hard wired. The network can include one or more of a wireless personal area network (WPAN), wireless local area network (WLAN), wireless metropolitan area network (WMAN), wireless wide area network (WWAN) and mobile networks. The network can be configured to operate the pump and receive data and inputs remotely, e.g., data and inputs from position sensors and radiation dosimeters or other internet of things (IOT) devices configured to be used in the medical procedures. The use of the position sensors allows the system to be configured automatically, e.g., raising or retracting the radiation shields automatically based on the location of the medical personnel in proximity to one or more of the radiation shields and/or surgical table.

In one embodiment, the control unit is configured to individually control the one or more extendable radiation shield sections by allowing for expansion in a vertical orientation. The radiation shield assembly includes a plurality of positionable radiation-shields, with each shield positioning mechanism that allows movement of tiles between a stacked (retracted) position and an extended position for overlapping coverage with adjacent stacks.

In one embodiment, the system is designed for automated operation based on user position or proximity, enhancing safety and usability in clinical settings by dynamically adjusting shield configurations without manual intervention with a input from a position over a network.

In one embodiment, the flexible radiation shields are constructed with a laminate structure including an inside layer, an outside layer, and a radiation shielding core material arranged between the layers, providing a flexible, soft, and impermeable surface suitable for medical environments.

In one embodiment, the radiation shield includes a laminate structure including an outer layer, an inner layer and a middle layer arranged between the outer layer and the inner layer. The middle layer includes a radiation shielding material or core material. The method includes attaching the layers together, e.g., one or more a chemical welding, solvent bonding, radio-frequency (RF) welding, heat welding, laser welding, stiches, weaving, stapling, crimping and combinations of the same and the like. Other suitable techniques, e.g., fastening, zipping, adhering, fusing, tacking, seaming, hemming.

In one embodiment, the outer layer includes a material including one or more of a thermoplastic material, an urethane material, a vinyl material, a polyvinyl chloride (PVC) material, cloth material, and combinations of the same and the like. The inner layer includes a material including one or more of a thermoplastic material, a urethane material, a vinyl material, and a polyvinyl chloride (PVC) material, cloth material, and combinations of the same and the like.

In one embodiment, one of the radiation shielding core includes a material having a lead equivalence (LE) in a range from about 0.125 mm to about 1.0 mm or greater.

In one embodiment, the radiation shielding core material includes a radiation protection material, e.g., one or more sheets of material, constructed of a high atomic weight element on one layer and a lower atomic weight element on another layer. In another embodiment, the material is edge bilayer from Kemmetech having a construction of two distinct homogenous layers as one integral sheet. The material can be distributed evenly to as specially graded metal particles (e.g., lead/non-Lead) within the vinyl matrix of each layer and therefore a consistent level of protection. In one embodiment, one or more of the following can be used Kemmetech LE Edge Bilayer 0.175 Bilayer Low Lead, 0.25 LE Bi-Layer Low Lead, 0.35 LE Bi-Layer Low Lead, 0.5 LE Bi-Layer Low Lead, 0.175 0.25 LE Bi-Layer Lead Free, 0.250 LE Bi-Layer Lead Free, 0.35 LE Bi-Layer Lead Free, 0.50 LE Bi-Layer Lead Free, 0.125 LE Lightweight Lead, 0.167 LE Lightweight Lead, 0.175 LE Lightweight Lead, 0.250 LE Lightweight Lead, 0.350 LE Lightweight Lead, 0.125 LE Superlight Lead, 0.167 LE Superlight Lead, 0.175 LE Superlight Lead, 0.250 LE Superlight Lead, 0.350 LE Superlight Lead, 0.125 LE Lead Free, 0.167 LE Lead Free, 0.175 LE Lead Free, 0.250 LE Lead Free, 0.350 LE Lead Free combinations of the same and the like. In another embodiment, other lead-free and lead composite products, e.g., containing low Z (atomic number) materials either exclusively or in a mixed metal composite can be utilized.

In one embodiment, lead equivalent materials are shielding substances designed to provide radiation protection comparable to a specific thickness of pure lead. Because lead has a very high atomic number and density, it is one of the most effective materials at attenuating ionizing radiation such as X-rays and gamma rays. However, pure lead can be heavy, rigid, and toxic, which makes it less practical for certain medical and industrial applications.

In one embodiment, the lead equivalent materials include high atomic number elements (such as tungsten, bismuth, antimony, or tin) embedded in lightweight polymers, composites, or fabrics. These radiation shielding materials are engineered so that a given thickness provides the same shielding effect as a standard thickness of lead (for example, a “0.5 mm lead equivalent” apron blocks the same amount of scatter radiation as 0.5 mm of pure lead).

In some aspects the radiation shielding material includes a first material with a high atomic weight element and a second material with a lower atomic weight element.

In some aspects, the techniques described herein relate to a system, wherein the radiation shielding material includes one of a 0.125 mm lead equivalence (LE) material, 0.25 mm lead equivalence (LE) material, a 0.5 mm lead equivalence (LE) material, a 0.75 lead equivalence (LE) material, a 1.0 lead equivalence (LE) material, a 1.25 lead equivalence (LE) material, a 1.5 mm lead equivalence (LE) material, a 1.75 lead equivalence (LE) material, a 2.0 lead equivalence (LE) material, and combinations of the same and the like.

In some aspects, the radiation shielding material includes a lead equivalence (LE) in a range from about 0.125 mm to about 1.0 mm or greater.

In some aspects, the radiation shielding material includes two or more layers includes a first outer layer including a material with a first atomic weight and an inner layer including a material with a second atomic weight, wherein the first atomic weight is lower than the second atomic weight. In one embodiment, the first outer layer includes antimony and the inner layer includes bismuth.

In some aspects, the techniques described herein relate to a system, wherein the at least one radiation shield has a weight in a range of 2 pounds to about 5 pounds or greater.

In some aspects, the techniques described herein relate to a system, wherein the at least one radiation shield is flexible and foldable.

In some aspects, the techniques described herein relate to a system, wherein the at least one radiation shield is soft to touch.

Optionally and/or alternatively, one or more of the radiation shields can also be configured to have a bend near the top of the shield or bottom as desired, which is believed to further mitigate scatter radiation.

In some aspects, one or more of the pneumatic channel and radiation shield segment can include one or more stiffing elements having a predetermined geometry. The predetermined geometry can be any geometry, e.g., having bends, linear or non-linear shapes, different thicknesses, combinations of the same and the like.

In one embodiment, the stiffing element includes a region that is configured to bend an upper portion of the pneumatic channel at angle in range from about 1 degree to about 35 degrees or greater relative to a vertical axis. For example, the radiation shield that extends vertically can have portion that bends at predetermined angle relative to a vertical axis. In one embodiment, the bend may be at angle from about 1 degree to about 35 degree or greater relative to a vertical axis.

In one embodiment, the stiffing elements may incorporate metals or engineered materials such as annealed aluminum for lightweight rigidity and case of forming, Nitinol for shape memory or self-deploying features, and high-density polyethylene (HDPE) for chemical resistance and durability under repeated flexing. Additionally, polymeric materials such as nylon, polypropylene, or other engineering plastics may be employed. In yet other implementations, hybrid structures may be created from composites, layered textiles, or combinations of metal alloys with polymer skins to balance flexibility, predetermined geometries and load-bearing requirements.

In one embodiment, the stiffing element is configured to provide enhanced rigidity to the one or more adjustable radiation shield segments.

In some aspects, the techniques described herein relate to a system, further including a manifold configured to be in fluid communication with the pneumatic channel.

In some aspects, the techniques described herein relate to a system, wherein the one or more adjustable radiation shield segments is releasably coupled with an adhesive.

In some aspects, the techniques described herein relate to a system, further including one or more dosimeters configured to monitor cumulative radiation exposure.

In some aspects, the techniques described herein relate to a system, wherein the one or more dosimeters are releasably coupled or coupled to the at least one radiation shield.

In some aspects, the techniques described herein relate to a system, wherein the one or more dosimeters are releasably coupled to one or more of a medical personal and the at least one radiation shield.

In some aspects, the techniques described herein relate to a system, wherein the one or more dosimeters are in communication over a network.

In some aspects as described herein, the system can further include radiation protective garments including aprons, thyroid shields, and shields. In one embodiment, the system can further include radiation protective garments including aprons, thyroid shields, and shields. as described with reference to U.S. application Ser. Nos. 29/949,882, 29/949,883, 29/949,886, 29/949,888, 29/949,890, and 63/734,336, each of which is hereby incorporated by reference as if fully set forth herein. Moreover, the system may also include a wearable weight support as described with reference to U.S. Pat. Nos. 10,729,195 and 11,627,795 as if fully set forth herein and including features such as a support system that transfers the weight of heavy garments that rely on shoulder support, off of the shoulders to the hips of the user including back support can be made from radio-opaque materials, lumbar support and results in less body heat containment through natural venting, easily adjustable to any body type and allows the wearer to move more freely in their work environment and can be used to transfer the weight of a backpack or other systems which rely on shoulder support to function.

In some aspects, the techniques described herein relate to a system, further including a surgical table extension, including: a generally planar body having a top surface and a bottom surface; and a recess in the generally planar body, the recess being open on one side and bounded on three sides by edge portions of the generally planar body including the top surface, the bottom surface and side surfaces extending from the top surface and the bottom surface, the recess is configured to receive at least a portion of the surgical table.

In some aspects, the techniques described herein relate to a system, further including a surgical table extension, including: a generally planar body having a top surface and a bottom surface configure to be attached mechanism to a portion the surgical table in order to increase a circumference of the surgical table.

In some aspects, the techniques described herein relate to a system, wherein the surgical table extension includes one or a composite material, a carbon fiber material, a fiber-glass material, a plastic material, a thermoplastic material and combinations of the same and the like.

In some aspects, the techniques described herein relate to a system, wherein the extension unit includes a first component configured to be rotationably attached to the one or more attachment holes and a second component configured to be rotationably attached to the first component.

In some aspects, the techniques described herein relate to a system, wherein the first component includes a protrusion configured to releasably engage one of the one or more attachment holes.

In some aspects, the techniques described herein relate to a system, wherein the second component is movably attached to the first component with a hinge mechanism.

In some aspects, the techniques described herein relate to a system, wherein the first component and second component when attached to the surgical table extension are configured to rotate and receive and support an arm of a patient.

In some aspects, the techniques described herein relate to a system, wherein the surgical table extension includes one or a composite material, a carbon fiber material, a fiber-glass material, a plastic material, a thermoplastic material and combinations of the same and the like.

In some aspects, the techniques described herein relate to a system, further including a bracket unit is configured to releasably mount to a rail on the surgical table.

In some aspects, the techniques described herein relate to a system, wherein the bracket unit is configured to lock into place with one or more locking mechanisms.

In some aspects, the techniques described herein relate to a system, wherein the bracket unit is configured to be attached to one or more of a working table, a light fixture, a medical apparatus, an extension arm, and combinations of the same.

In one embodiment, the system includes a bracket is configured to slidably and vertically engage a rail of a surgical table. The bracket is configured to be vertically adjustable on a rail of surgical table and horizontal adjustable on the rail of the surgical table. In addition, the bracket is configured to receive one or more of a table, a first extension unit, a second extension unit 2904, a tray or other surgical apparatus. The vertical adjustment is configured to allow a medical personal to position the attachment flush (at the same level) with the surgical table, higher or lower as compared to the surgical table or surgical table extension.

In one embodiment, a method of operating the apparatus includes providing at least one radiation shield configured to be connected to a surgical table with an attachment mechanism. The at least one radiation shield includes one or more adjustable radiation shield segments coupled or releasably coupled to the at least one radiation shield, a pump in fluid communication with the one or more adjustable radiation shield segments including a pneumatic channel configured to at least partially inflate the pneumatic channel such that at least a first portion of the at least one radiation shield extends vertically above at least a portion of the surgical table and where a second portion of radiation shield is configured to extend below the surgical table; and a controller configured to activate the pump. The method includes attaching the at least one radiation shield to the surgical table and activating the pump to partially inflate the pneumatic channel and raise at least the first portion of the at least one radiation shield vertically above a portion of the surgical table.

Reference will now be made in detail to an embodiment of the present invention, example of which is illustrated in the accompanying drawings.

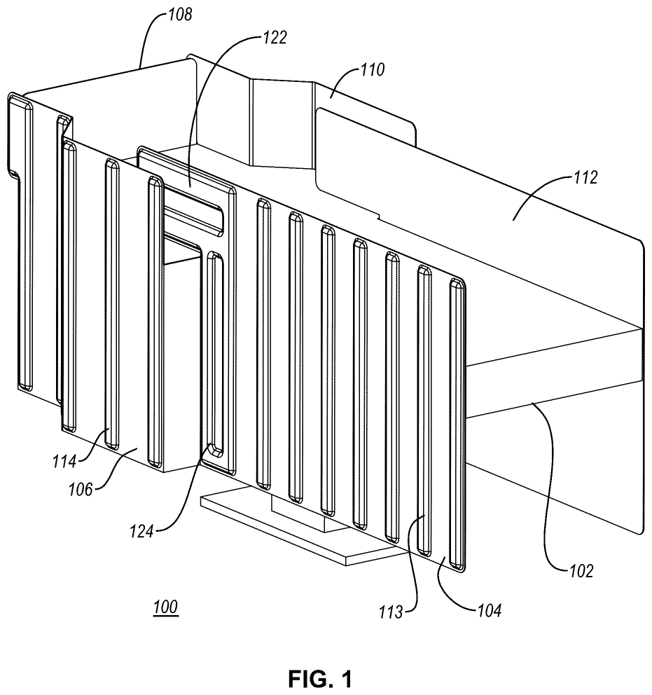

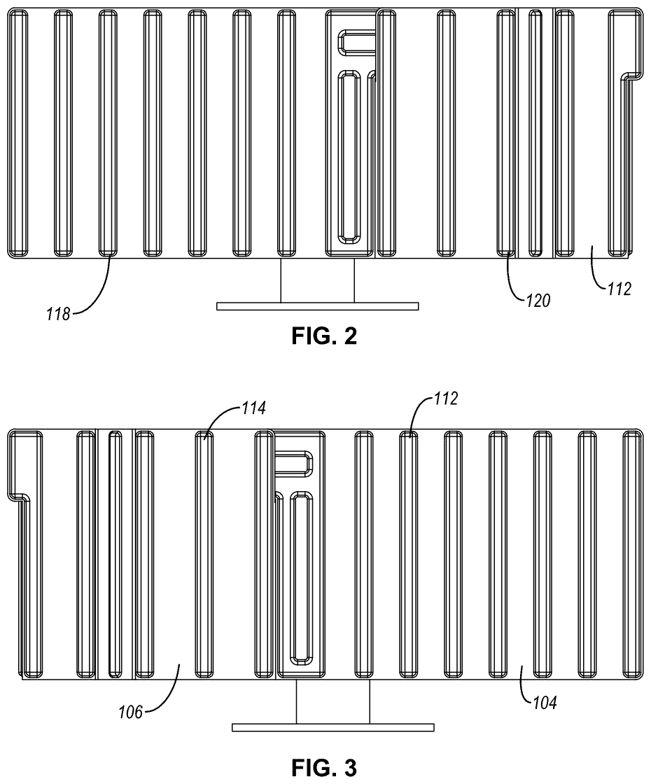

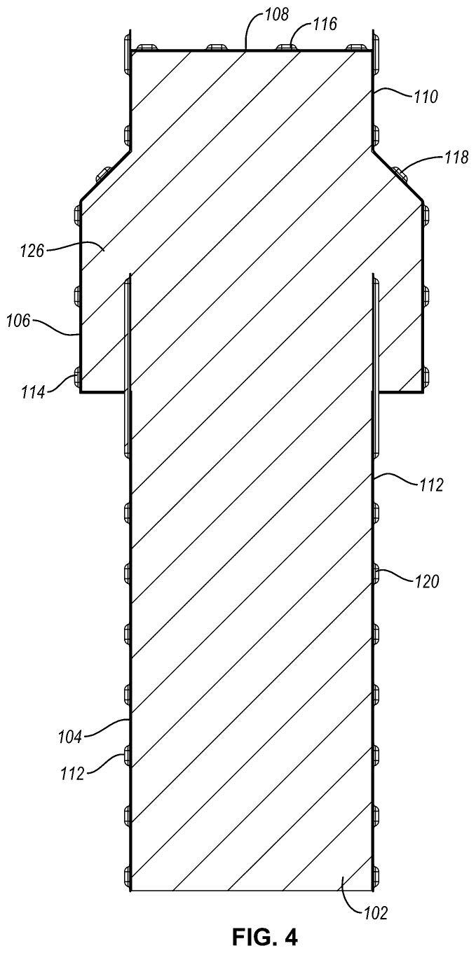

FIG. 1 illustrates an exemplary perspective view of a radiation shielding system arranged on a surgical table in an extended orientation according to an embodiment of the invention. FIG. 2 illustrates an exemplary left side view of FIG. 1 according to an embodiment of the invention. FIG. 3 illustrates an exemplary right side view of FIG. 1 according to an embodiment of the invention. FIG. 4 illustrates a exemplary top view of FIG. 1 according to an embodiment of the invention.

Referring to FIGS. 1-4, the radiation shielding system is generally depicted with reference to number 100. The radiation shielding system 100 is configured to reduce radiation, e.g., scatter radiation, from medical personal. The shielding system 100 includes one more extendable radiation shields attached to surgical table 102. In this embodiment, the plurality of flexible radiation shields includes flexible radiation shield 104 and flexible radiation shield 106 attached to a first side of the surgical table 100, flexible radiation shield 106 attached to a top of the surgical table 102, flexible radiation shield 108 and a flexible radiation shield 110 attached to the surgical table 102. Optionally and/or alternatively, the size and number of flexible radiation shields may vary, e.g., three on one side or greater.

In this embodiment, each of the plurality of flexible radiation shields includes one or more adjustable radiation shield segments. That is, the first flexible radiation shield 104 includes one or more adjustable radiation shield segments 113, flexible radiation shield 106 includes one or more adjustable radiation shield segments 114, flexible radiation shield 108 includes one or more adjustable radiation shield segments 116, flexible radiation shield 110 includes one or more adjustable radiation shield segments 118, and flexible radiation shield 112 includes one or more adjustable radiation shield segments 120. In this embodiment, the one or more adjust radiation shield segments are integral with each of the flexible radiation shields. Optionally and/or alternatively, each of the radiation shield segments can be releasably attached or permanently coupled to a surface of the flexible radiation shields as described herein.

In the embodiment, each of the radiation shield segments includes one or more pneumatic channels configured to hold pressurized air or a another fluid. The pneumatic channels can be in any geometric configuration, straight, curved, bent and can share a channel. For example, pneumatic channel 122 and pneumatic channel 124 are shared and bent at about a 90 degree angle.

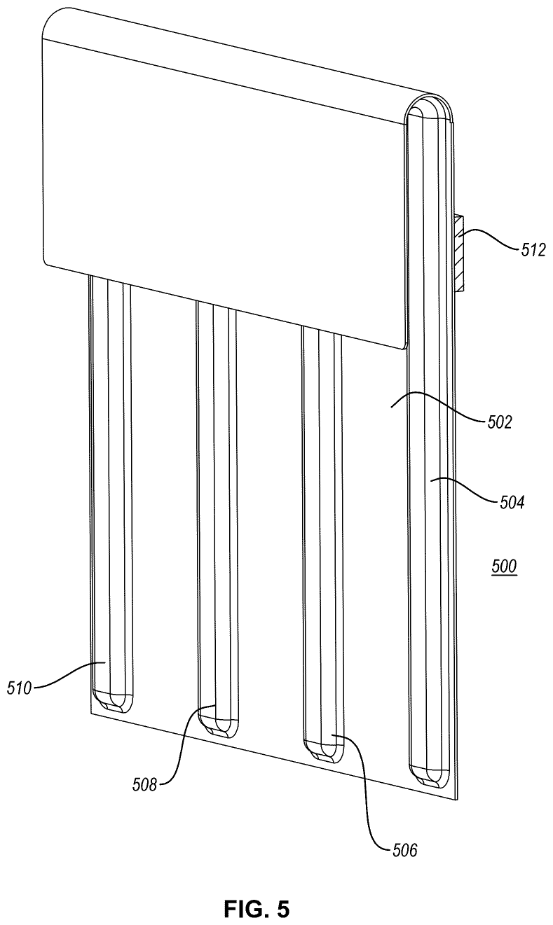

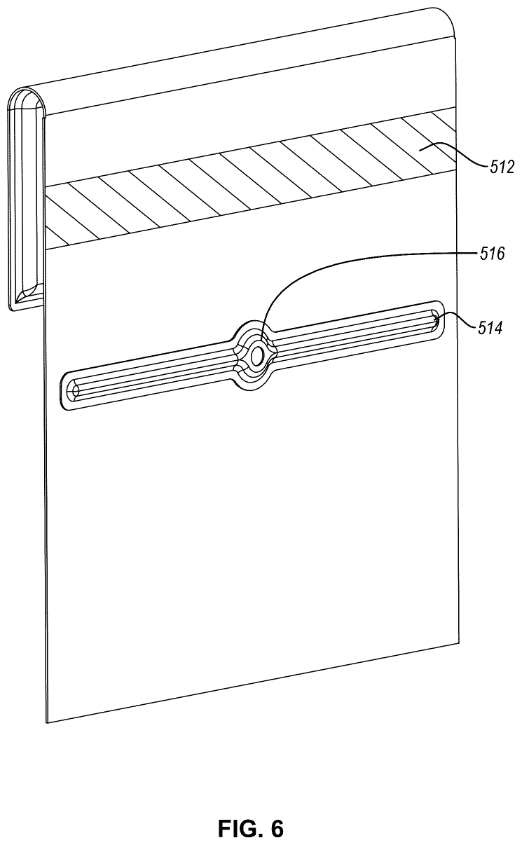

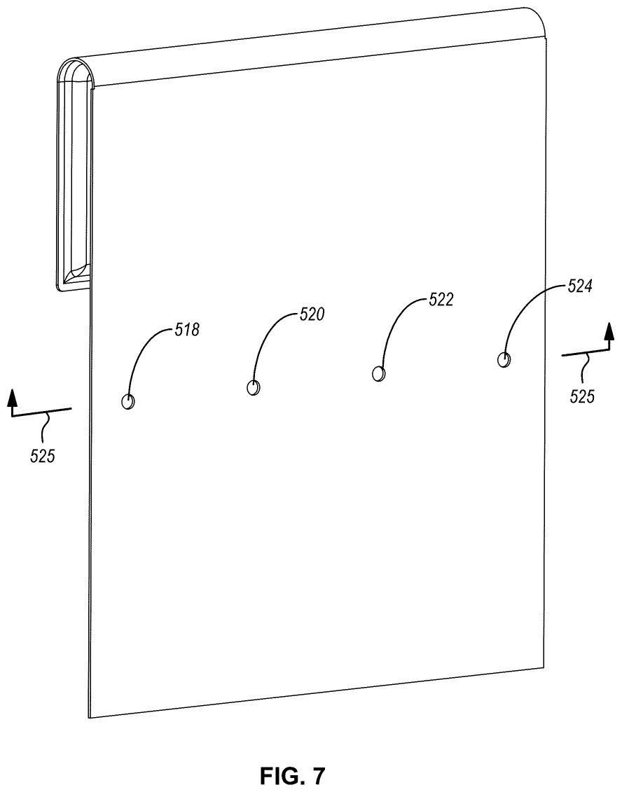

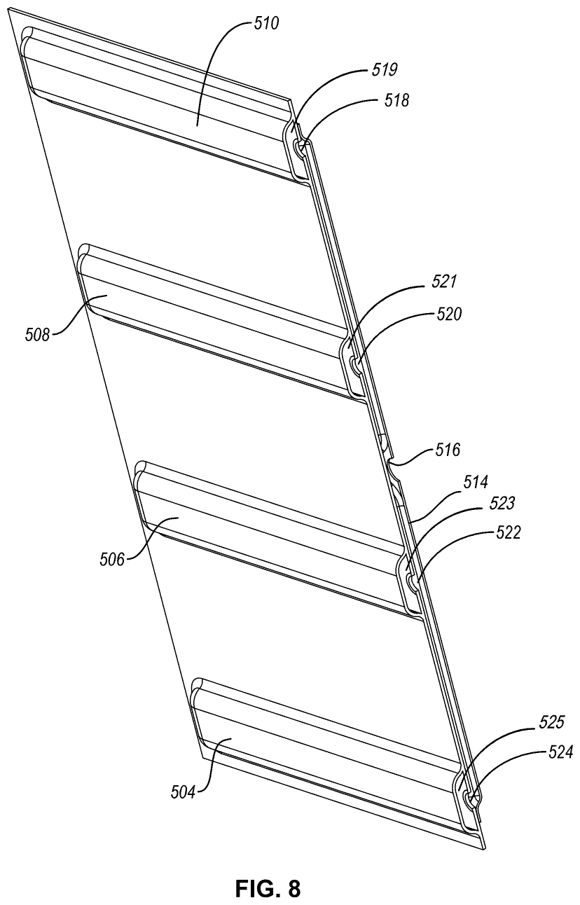

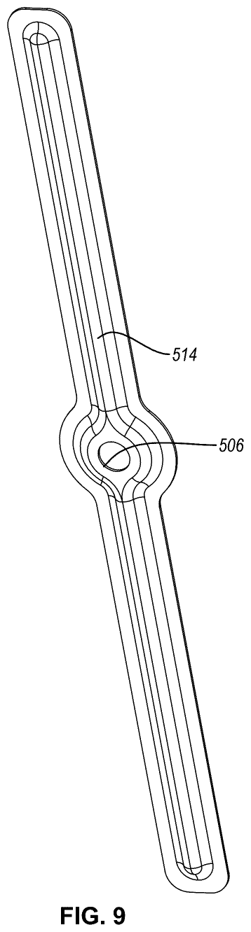

FIG. 5 illustrates an exemplary partial perspective front view of a flexible radiation shield of FIG. 1 in a non-extended orientation with a manifold according to an embodiment of the invention. FIG. 6 illustrates an exemplary rear perspective front view of FIG. 5 in a non-extended orientation with a manifold according to an embodiment of the invention. FIG. 7 illustrates an exemplary rear perspective front view of FIG. 5 in a non-extended orientation without a manifold according to an embodiment of the invention. FIG. 8 illustrates an exemplary cross-sectional view of a FIG. 7 along line 525 to 525. FIG. 9 illustrates an exemplary perspective view of a manifold of FIG. 5 according to an embodiment of the invention.

Referring now FIGS. 5-9, the partial view of the radiation shield assembly 104 is shown with reference to number 500. The radiation shield assembly 500 includes a radiation shield 502 with radiation shield segment 504, radiation shield segment 506, radiation shield segment 508, and radiation shield segment 510 in a non-inflated state or retracted configuration. In this configuration, the radiation shield 502 is attached to a portion of surgical table 102 with an attachment mechanism 512. In this embodiment, the attachment mechanism 512 includes a hook and loop attachment that corresponds to one on the surgical table 102 or surgical table extension. The upper portion hangs over in this non-inflated state or retracted configuration.

Each of the radiation shield segments 504, 506, 508, and 510, include one or more pneumatic air channels configured to be pressured from 1 psi to 50 psi or greater with a pressurization device, e.g., pump. Again, each of the flexible radiation shield can be individually controlled, controlled together or controlled in any predetermined scheme, e.g., one at time, two at time, three at time, etc., with a control unit where each of the flexible radiation shields are configured to raise and lower each above the surgical table to a predetermined height. In one embodiment, the control unit is in communication with controller configured to activate the pump. In this embodiment, the pump has a line, e.g., air line, connected to a manifold 514 having an inlet 516. The manifold 514 is coupled to the radiation shield 502 and allows pressurized air to be received by each inlet 518, inlet 520, inlet 522 and inlet 524 simultaneously. The inlet 516 can be coupled to another manifold with controllable valves and coupled to a pump or directly coupled to a pump. In this embodiment, the radiation shield segment 504 includes an pneumatic channel 525, radiation shield segment 506 includes an pneumatic channel 523, radiation shield segment 508 includes an pneumatic channel 521, and radiation shield segment 510 includes an pneumatic channel 519.

In this embodiment, a pump in communication with the inlet 516 of the manifold 514 pressurizes the pneumatic channels 519, 521, 523, and 525 to predetermined pressure, e.g., 3-10 psi to extend the flexible radiation shield 502 above the surgical table. The radiation shield 502 can be sized and attached to the surgical table so it can extend vertically above the surgical table in a range from about 3 inches to 18 inches or greater. In a preferred embodiment, the extension of one or more of the radiation shields above the surgical table is about 3 inches to about 12 inches or less. The flexible radiation shield 502 is also sized and positioned on the surgical table to extend below the surgical table to length of about three inches or more above the floor.

Optionally and/or alternatively, the pump in communication with manifold is configured for rapid deflation by applying a negative pressure to each of the pneumatic channels 519, 521, 523 and 525 via the manifold 514, thereby enabling quick retraction of the radiation shield segment 502. A negative pressure is not required to retract the radiation shield 502, rather the positive pressure can simply be released and the shield 502 will retract on its own under ambient conditions. The vacuum pressure can be in a range from about 10⁻³ to 10⁻⁹ Torr with the pump and can be utilized for rapid retraction, e.g., in emergency situations.

Optionally and/or alternatively, each of the radiation shields can be configured with a pouch, cavity, or void configured to receive an inflatable bag or alternative expandable structure may be formed as a defined compartment integrated into or adjacent to a supporting surface, panel, or frame. The compartment can be constructed from flexible or semi-rigid material layers, for example textile laminates, coated fabrics, elastomers, or composite sheets, joined by stitching, welding, adhesive bonding, or thermal sealing to produce a sealed or semi-sealed enclosure. In this configuration, the dimensions of the pouch can be sized to accommodate the inflatable bag in its deflated state while providing sufficient clearance and guidance during expansion.

In one embodiment, the pouch includes a defined mouth or aperture through which the inflatable structure may be inserted, anchored, or deployed. This opening can be reinforced with grommets, rigid frames, or molded rims to prevent tearing during repeated inflation cycles. Internal seams or gussets may be incorporated to guide the unfolding of the bag and to ensure predictable orientation upon deployment. The pouch walls may incorporate pleats, folds, or stretchable regions to enable controlled expansion without material failure. Moreover, optional fastening or retention features, such as hook-and-loop patches, snaps, or drawcords, may be integrated to secure the inflatable bag in place prior to activation. Additionally, fluid conduits, valves, or ports may be routed through or alongside the pouch wall to facilitate inflation via compressed air, inert gas, or liquid, with seals or gaskets positioned to prevent leakage. The geometry of the void may be rectangular, cylindrical, or contoured, depending on the shape of the inflatable structure and the intended deployment environment. By combining flexible containment with structural reinforcement, the pouch provides a reliable housing for the inflatable bag that preserves compact storage while enabling rapid, repeatable deployment under load or pressure.

Optionally and/or alternatively, one or more of the radiation shields can also be configured to have a bend near the top of the shield or bottom as desired, which is believed to further mitigate scatter radiation. In some aspects, one or more of the pneumatic channel and radiation shield segment can include one or more stiffing elements having a predetermined geometry. The predetermined geometry can be any geometry, e.g., having bends, linear or non-linear shapes, different thicknesses, combinations of the same and the like.

In one embodiment, the stiffing element includes a region that is configured to bend an upper portion of the pneumatic channel at angle in range from about 1 degree to about 35 degrees or greater relative to a vertical axis. For example, the radiation shield that extends vertically can have portion that bends at predetermined angle relative to a vertical axis. In one embodiment, the bend may be at angle from about 1 degree to about 35 degree or greater relative to a vertical axis. In one embodiment, the stiffing elements can includes a material including an annealed aluminum, nitinol, other materials described herein and combinations of the same. In one embodiment, the stiffing element is configured to provide enhanced rigidity to the one or more adjustable radiation shield segments.

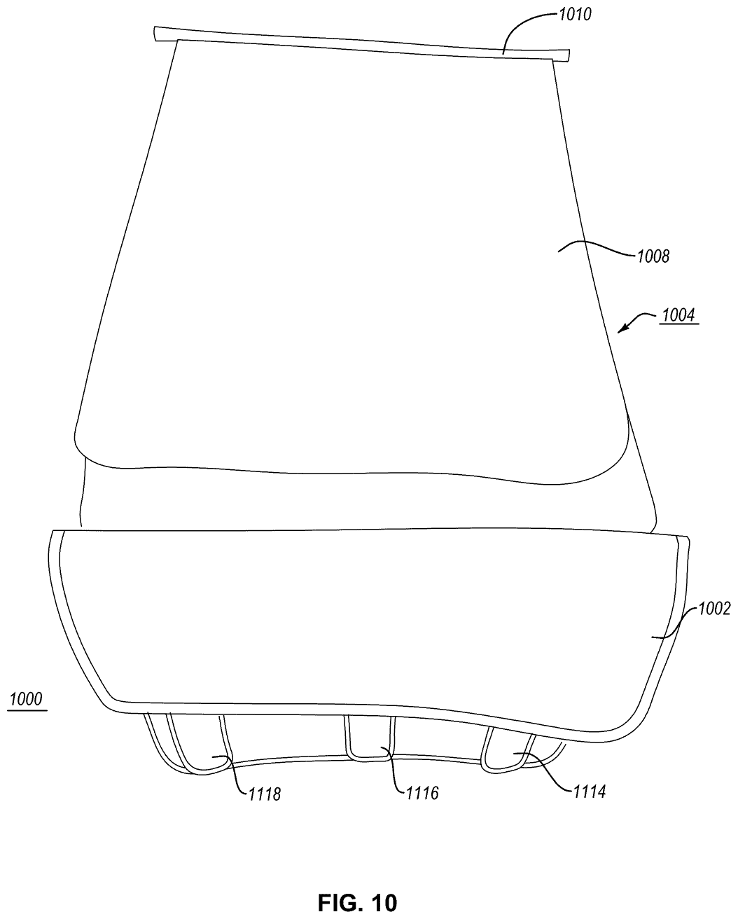

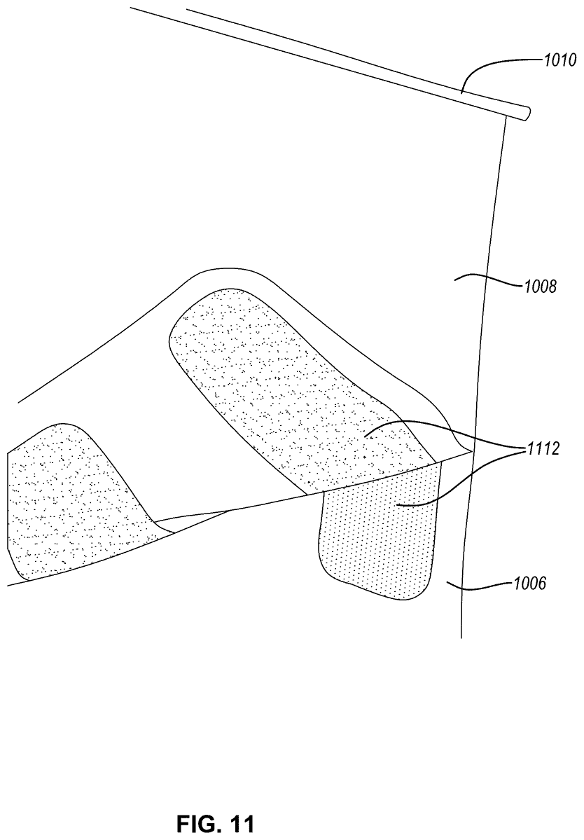

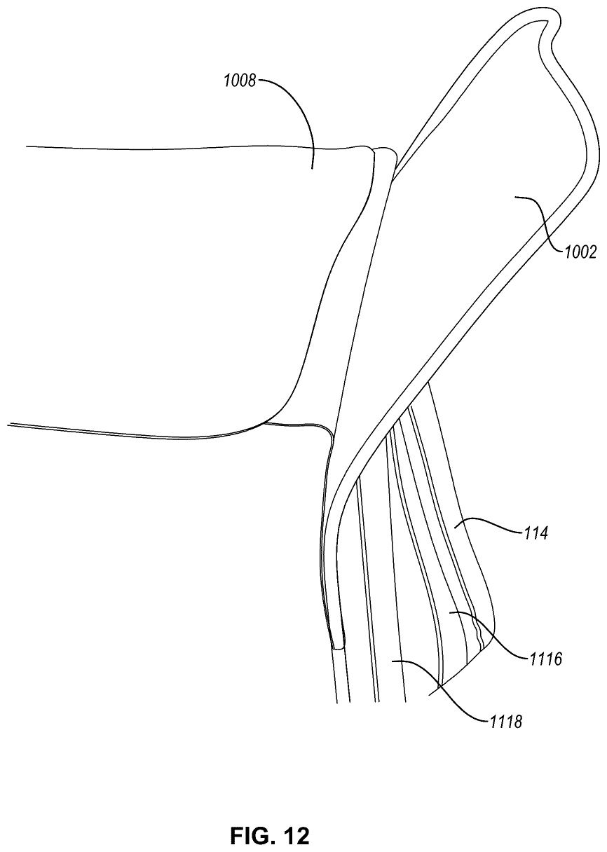

FIG. 10 illustrates an exemplary perspective front view of a system including a flexible radiation shield in a non-extended orientation according to an embodiment of the invention. FIG. 11 illustrates a partial top view of FIG. 10 according to an embodiment of the invention. FIG. 12 illustrates a perspective side view of FIG. 10 according to an embodiment of the invention.

Referring to FIGS. 10-12, the system configured to reduce scatter radiation around a surgical table is generally depicted with reference to number 1000. The system 1000 includes one or more radiation shields attached to a surgical table with a support member 1004. The support member 1004 includes a first portion 1006 attached to a first radiation shield 1002 and second portion 1008 attached to a second radiation shield 1010. In this embodiment, the attachment is done with a hook and loop mechanism such as VELCRO, but other attachment mechanism 1112 may be utilized, e.g., magnets, snaps, zippers, combinations of the same and the like. The attachment mechanism may be releasable or non-releasable.

The radiation shield 1002 includes an adjustable radiation shield segment 1114, an adjustable radiation shield segment 1116, and an adjustable radiation shield segment 1118. Each of the adjustable radiation shield segments 1114, 1116, and 1118 include one or more pneumatic channels.

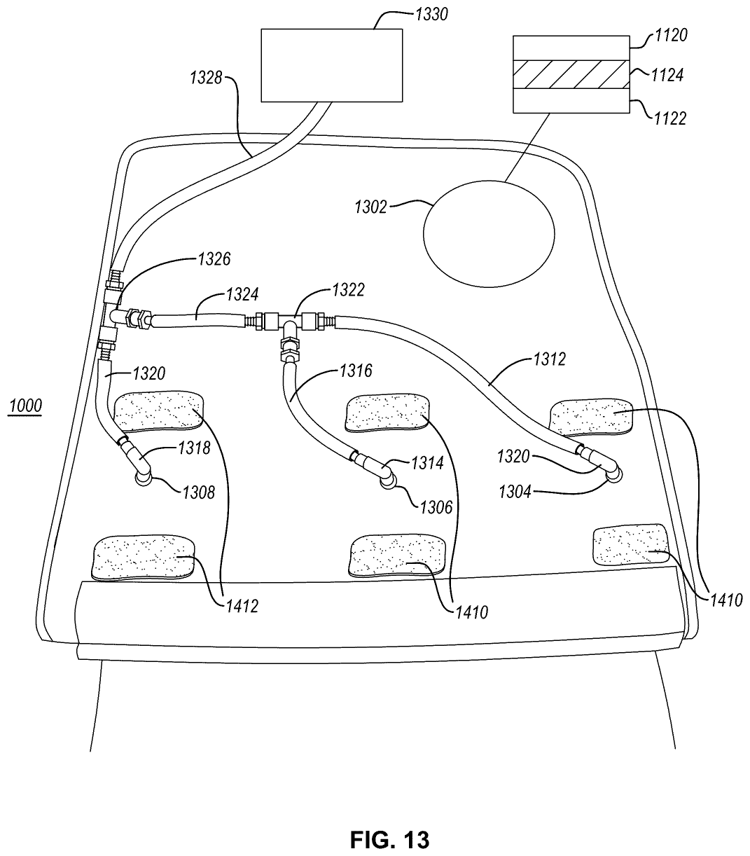

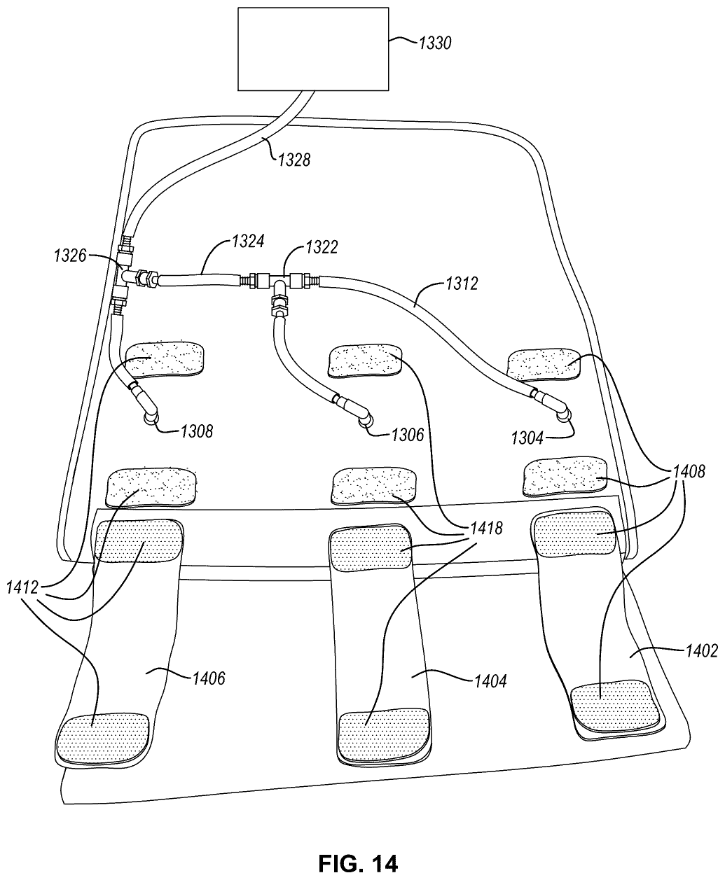

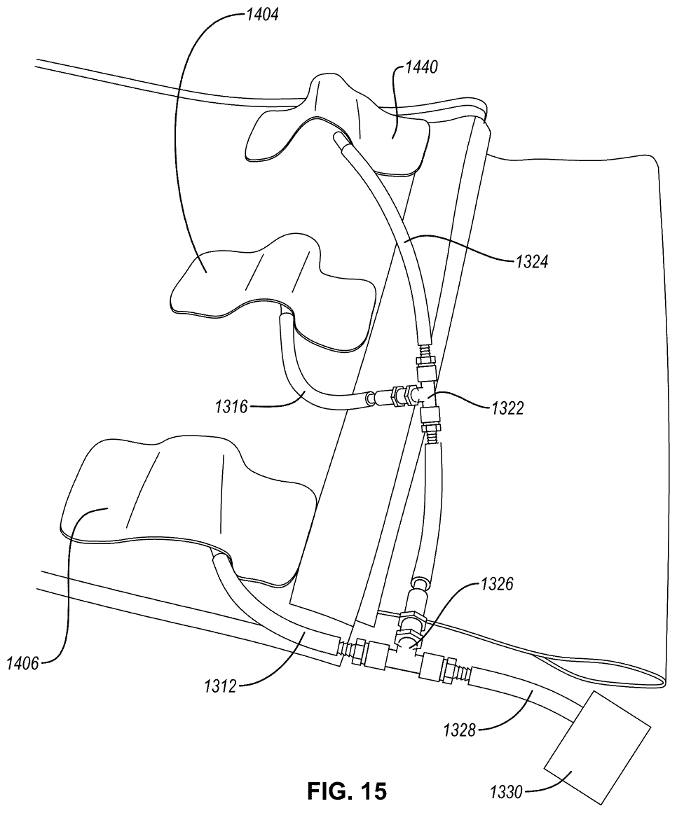

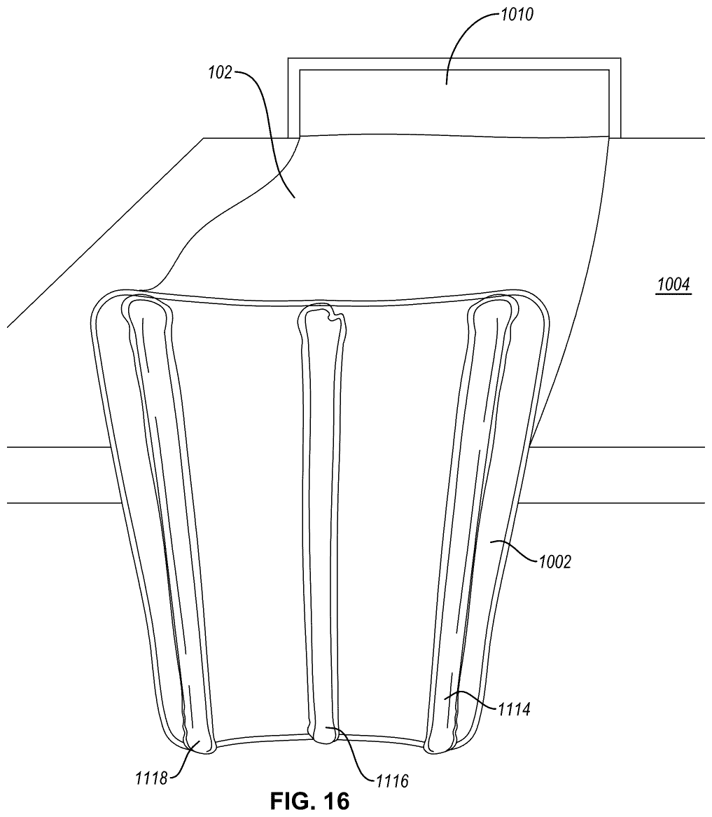

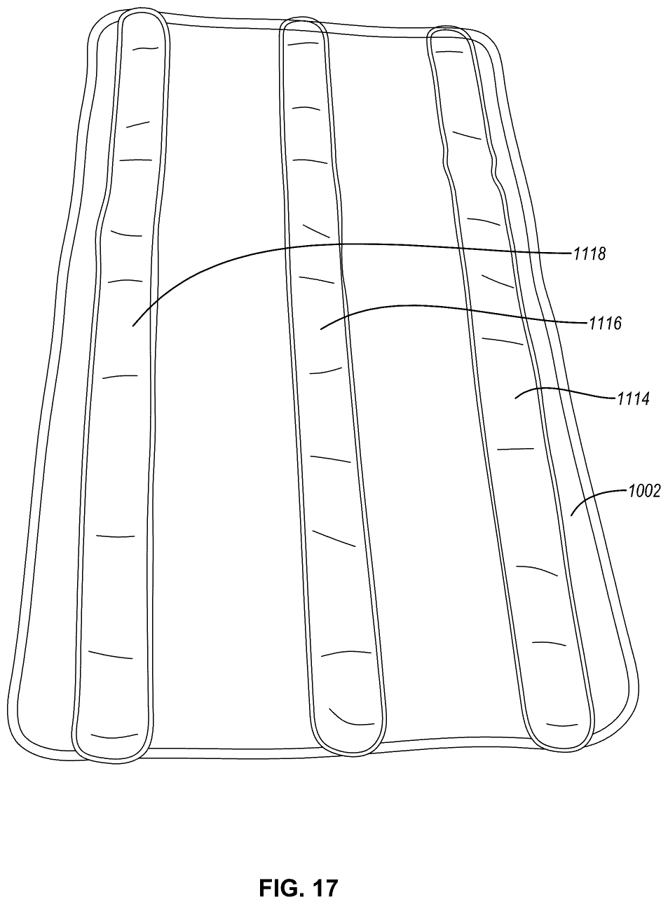

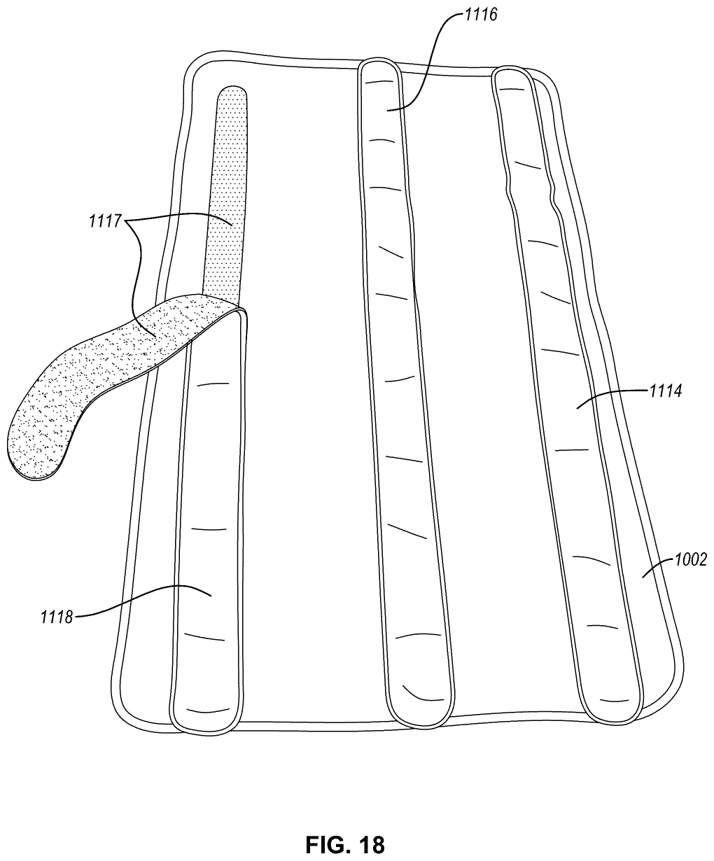

FIG. 13 illustrates a perspective inner view of the flexible radiation shield of FIG. 10 without pneumatic channel port covers according to an embodiment of the invention. FIG. 14 illustrates a perspective inner view of the flexible radiation shield of FIG. 10 with pneumatic channel port covers in an uninstalled configuration according to an embodiment of the invention. FIG. 15 illustrates a perspective inner view of the flexible radiation shield of FIG. 10 with pneumatic channel port covers installed according to an embodiment of the invention. FIG. 16 illustrates a perspective side otter view of the flexible radiation shield of FIG. 10 in an expanded configuration according to an embodiment of the invention. FIG. 17 illustrates a perspective view of the flexible radiation shield of FIG. 10 according to an embodiment of the invention. FIG. 18 illustrates a perspective view of FIG. 10 with the adjustable radiation shield segments in a partially detached configuration according to an embodiment of the invention.

Referring to FIGS. 13-18, the system 1000 includes the adjustable radiation shield 1002 that is flexible and soft and constructed with a laminate structure as shown by cross-section 1302 including an inside layer 1120, an outside layer 1122, and a radiation shielding core 1124 material arranged between the layers, providing a flexible, soft, and impermeable surface suitable for medical environments. The radiation shielding core 1124 can include more than one layer and constructed from any of the materials such that it has a lead equivalence (LE) in a range from about 0.125 mm to about 1.0 mm or greater.

The outer layer 1120 and inner layer 1122 can also be more than one layer and include one or more of a thermoplastic material, an urethane material, a vinyl material, a polyvinyl chloride (PVC) material, cloth material, and combinations of the same and the like.

In this embodiment, there is no manifold associated with the inputs to each of the pneumatic channels. Rather, in this embodiment, each pneumatic channel includes a direct airline in communication with a pump, manifold and valves system. By way of illustrative example, input 1304 is an input to one or more pneumatic channels associated with the adjustable radiation shield segment 1114, input 1306 is an input to one or more pneumatic channels associated with the adjustable radiation shield segment 1116, and input 1308 is an input to one or more pneumatic channels associated with the adjustable radiation shield segment 1118. Input 1304 is coupled to a connector 1320, e.g., elbow connector or other type of connector and an airline segment 1312. Input 1306 is coupled to a connector 1314, e.g., elbow connector or other type of connector and an airline segment 1316. Input 1308 is coupled to a connector 1318, e.g., elbow connector or other type of connector and an airline segment 1320. Airline segment 1312 is coupled to airline segment 1316 and with a connector 1322. Connector 1322, in this embodiment, is a T-type connector and also coupled to airline segment 1324. Airline segment is coupled to airline segment 1320 and airline segment 1328 with a connector 1326, e.g., T-type connector. Airline segment 1328 is in communication one or more a controllable air distribution source 1330. In this embodiment, the controllable air distribution source includes a manifold, controllable valves and a pump. A control unit and controller are in communication with the controllable air distribution source.

Referring to FIGS. 13-18, input 1304 has a protective removable cover 1402 configured to be releasably attached to the radiation shield 1002 with an attachment mechanism 1408, e.g., hook and loop attachment mechanism or other attachment mechanism as described herein. Input 1306 has a protective removable cover 1404 configured to be releasably attached to the radiation shield 1002 with an attachment mechanism 1410, e.g., hook and loop attachment mechanism or other attachment mechanism as described herein. Input 1308 has a protective removable cover 1406 configured to be releasably attached to the radiation shield 1002 with an attachment mechanism 1412, e.g., hook and loop attachment mechanism or other attachment mechanism as described herein.

Referring to FIG. 16, the system 1000 includes a first radiation shield 1002 and second radiation shield 1010 in an expanded configuration. Now referring to FIGS. 17-18, the radiation shield 1002 includes an adjustable radiation shield segment 1118 releasably attached to an outside portion of the radiation shield 1002 with an attachment mechanism 1117. In this embodiment, the attachment mechanism 1117 is done with a hook and loop mechanism such as VELCRO, but other attachment mechanism may be utilized, e.g., magnets, snaps, zippers, combinations of the same and the like. Optionally and/or alternatively, the adjustable radiation shield segments may be permanently attached to the radiation shield 1002 by one or more chemical welding, solvent bonding, radio-frequency (RF) welding, heat welding, laser welding, stiches, weaving, stapling, crimping and combinations of the same and the like.

Optionally and/or alternatively, one or more of the radiation shields can also be configured to have a bend near the top of the shield or bottom as desired, which is believed to further mitigate scatter radiation. In some aspects, one or more of the pneumatic channel and radiation shield segment can include one or more stiffing elements having a predetermined geometry. The predetermined geometry can be any geometry, e.g., having bends, linear or non-linear shapes, different thicknesses, combinations of the same and the like.

In one embodiment, the stiffing element includes a region that is configured to bend an upper portion of the pneumatic channel at angle in range from about 1 degree to about 35 degrees or greater relative to a vertical axis. For example, the radiation shield that extends vertically can have portion that bends at predetermined angle relative to a vertical axis. In one embodiment, the bend may be at angle from about 1 degree to about 35 degree or greater relative to a vertical axis. In one embodiment, the stiffing elements can includes a material including an annealed aluminum, nitinol, and combinations of the same. In one embodiment, the stiffing element is configured to provide enhanced rigidity to the one or more adjustable radiation shield segments.

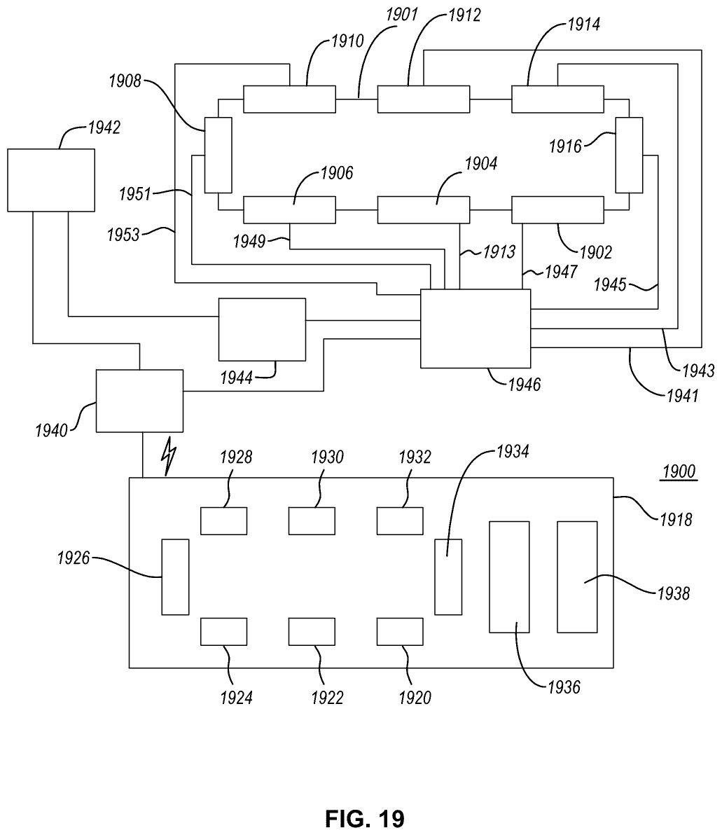

Referring to FIG. 19, the system is generally depicted with reference to number 1900. The system 1900 is configured to reduce scatter radiation around a surgical table during a medical procedure. The system includes radiation shields arranged around a perimeter of a surgical table 1901. The number and positioning can be determined by the type of procedure and configured to maximize protection of the medical personal. In this embodiment, the system includes a flexible radiation shields as described as follows a first radiation shield 1902, a second radiation shield 1904, and a third radiation shield 1906 on a first side of the surgical table 1901. A fourth radiation shield 1908 at the head portion of the surgical table 1901. A fifth radiation shield 1910, six radiation shield 1912, a seventh radiation shield 1914 on an opposite side of the surgical table 1901. Optionally, an eight radiation shield 1916 is positioned at a foot of the surgical table 1901.

Optionally and/or alternatively, the surgical table can include a surgical table extension as described herein. Each of the radiation shields includes on or more adjustable radiation shield segments including one or more pneumatic channels as described herein. Each of the pneumatic channel is in communication with a pressurization source, e.g., pump, configured to extend and retract each of the radiation shields in a predetermined manner. The system 1900 includes a control unit 1918. The control unit 1918 can be a push button, touch screen, combination of the same and the like. The control unit 1918 is configured to extend or retract each of the radiation shields independently, any scheme, e.g., one or more shields, and/or all at one time. In this embodiment, the control unit 1918 has a button 1920 corresponding to the first radiation shield 1902 where activation of button extends or retracts the upper portion the radiation shield 1902. Button 1922 is configured to correspond to the second radiation shield 1904 where activation of button extends or retracts the upper portion the second radiation shield 1904. Button 1924 is configured to correspond to the third radiation shield 1906 where activation of button extends or retracts the upper portion the third radiation shield 1906. Button 1926 is configured to correspond to the fourth radiation shield 1908 where activation of button extends or retracts the upper portion the fourth radiation shield 1908. Button 1928 is configured to correspond to the fifth radiation shield 1910 where activation of button extends or retracts the upper portion the fifth radiation shield 1910. Button 1930 is configured to correspond to the sixth radiation shield 1912 where activation of button extends or retracts the upper portion the sixth radiation shield 1912. Button 1932 is configured to correspond to the seventh radiation shield 1914 where activation of button extends or retracts the upper portion the seventh radiation shield 1914. Button 1934 is configured to correspond to the eight radiation shield 1916 where activation of button extends or retracts the upper portion the eight radiation shield 1916.

In this embodiment, any of the buttons can be physical buttons, touch screen buttons, combinations of the same or the like and arranged in any manner graphically. In a preferred embodiment, the buttons are oriented (on touch screen or physical buttons) to mimic the spatial orientation of the shields around the surgical table. In this embodiment, button 1936 is configured to rapidly retract each of the radiation shields through use of vacuum pressure as described herein. In addition, button 1938 is configured to extend all of the radiation shields. The control unit 1918 can be hardwired or wirelessly in communication with the system. Additional buttons can be configured control one or more the radiation shields.

The system 1900 further includes a controller 1940 in communication with a pressurization source 1942, e.g., pump, and valve and manifold unit 1946. The system 1900 also includes a pressure regulator 1944 in communication with the pump and valve and manifold unit 1946. The valve and manifold unit 1946 is configured to operate each one of the radiation shields independently or in any predetermined scheme.

In this embodiment, the valve and manifold unit 1946 connects to the first radiation shield 1902 with line 1947, e.g., airline or channel. The valve and manifold unit 1946 connects to the second radiation shield 1904 with line 1913, e.g., airline or channel. The valve and manifold unit 1946 connects to the third radiation shield 1906 with line 1949, e.g., airline or channel. The valve and manifold unit 1946 connects to the fourth radiation shield 1908 with line 1951, e.g., airline or channel. The valve and manifold unit 1946 connects to the fifth radiation shield 1910 with line 1953, e.g., airline or channel. The valve and manifold unit 1946 connects to the sixth radiation shield 1912 with line 1941, e.g., airline or channel. The valve and manifold unit 1946 connects to the seventh radiation shield 1914 with line 1943, e.g., airline or channel. The valve and manifold unit 1946 connects to the eighth radiation shield 1945 with line 1945, e.g., airline or channel.

The control unit can be in communication over a network described herein. In addition, the control unit configured to retract the one or more extendable radiation shield sections or segments based on an input from a positioning sensor coupled to at least one of the one or more extendable radiation shield sections or segments and is configured to sense and react to positioning or proximity of a user or radiation source. The position sensor is in communication over the network and configured to detect a location of a medical personal in proximity to one of the radiation shields. The controller receives a predetermined distance and when the positioning sensor is within the predetermined distance it activates the pump to raise the one or more radiation shield sections or segments. In one embodiment, the dosimeter is also in communication over the network.

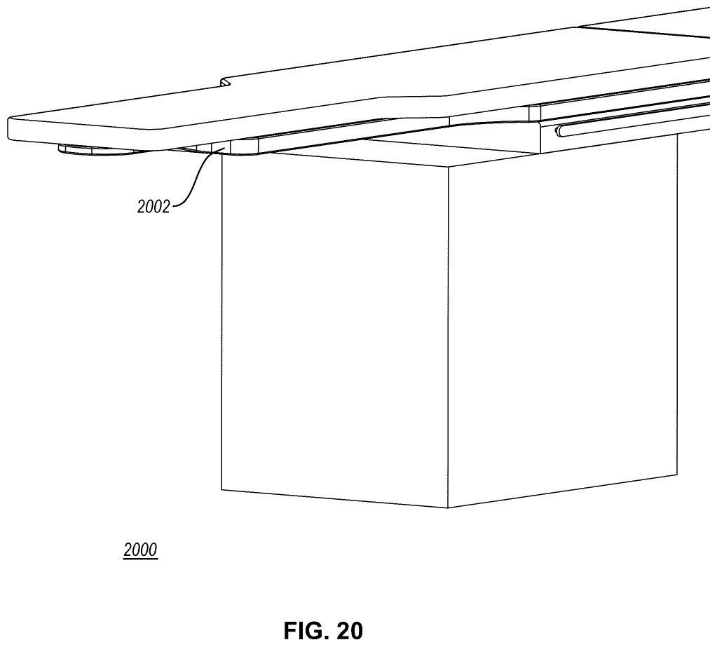

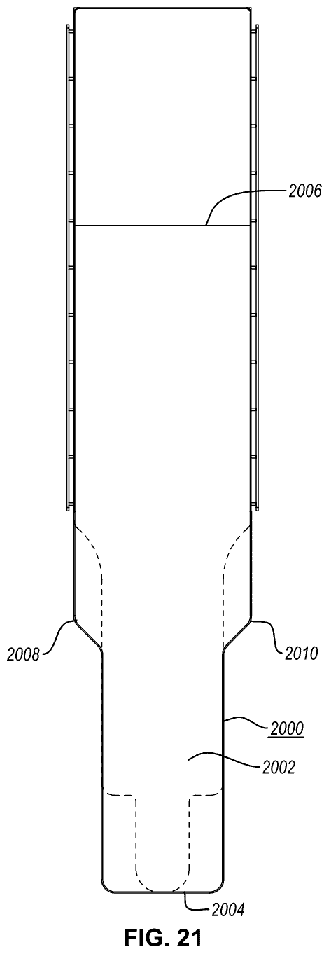

FIG. 20 illustrates a perspective side view of a table extension for a surgical table according to an embodiment of the invention. FIG. 21 illustrates a top view of FIG. 20 according to an embodiment of the invention.

Referring to FIGS. 20-21, a surgical table extension is generally depicted with reference to number 2000. The surgical table extension 2000 includes one piece extension configured to be attached to an upper portion of a standard surgical table 2002. Optionally, and/or alternatively, the surgical table extension 2000 can be made with two pieces that join together.

In this embodiment, the surgical table extension 2000 is configured to increase the circumference and/or perimeter of a surgical table 2002. The oversizing of the surgical table extension 2000 as compared to at least portions of surgical table 2002 is further configured to permit one more radiation shields to better cover C-arm and mitigate scatter radiation by allowing the radiation shields to extend below the table in a wider manner by increasing the circumference or perimeter of the surgical table 2002.

The surgical table extension 2000 includes any attachment mechanism as described to releasably attach one or more surgical shields to the a perimeter of the surgical table extension 2000. In a preferred embodiment, the attachment mechanism includes a hook and loop attachment mechanism that corresponding to a hook and loop attachment mechanism on the radiation shield. Of course, other attachment mechanisms can be utilized, e.g., snaps, magnets, clips and combinations of the same and the like.

Moreover, the surgical table extension 2000 is releasably attached to a top portion of a surgical table 2002 with a hook and loop attachment mechanism or other attachment mechanism described herein.

The surgical table extension 2000 includes a material that is substantially transparent to x-ray radiation or substantially a radiolucent material. In one embodiment, the surgical table extension includes one or a composite material, a carbon fiber material, a fiber-glass material, a plastic material, a thermoplastic material and combinations of the same and the like.

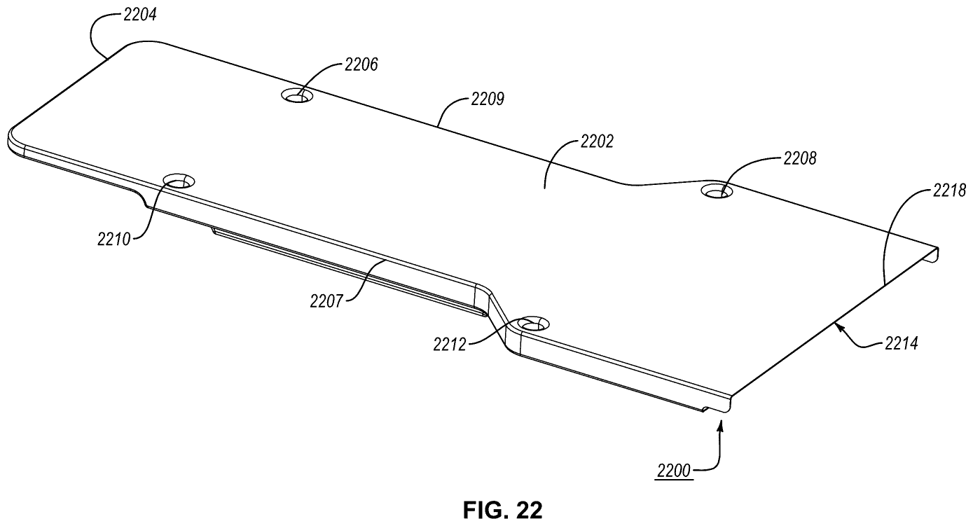

That surgical table extension 2000 includes a first end 2004 a spaced apart second end 2006, a first side 2008 and a second side 2010 spaced apart from the first side 2008. Optionally, and/or alternatively, the surgical table extension 2000 can include one or more recesses for receiving an extension as with references FIGS. 22-34 and elsewhere herein.

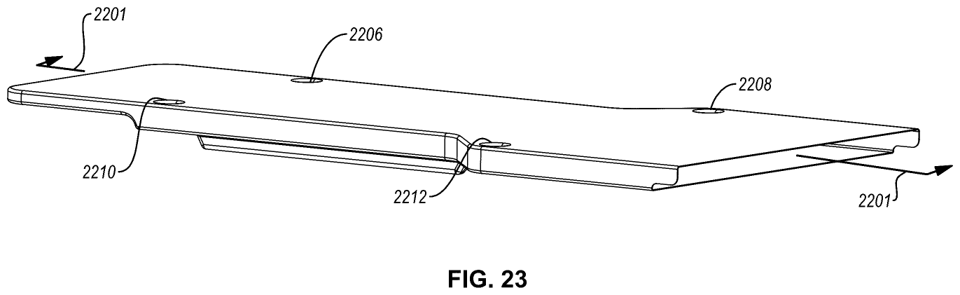

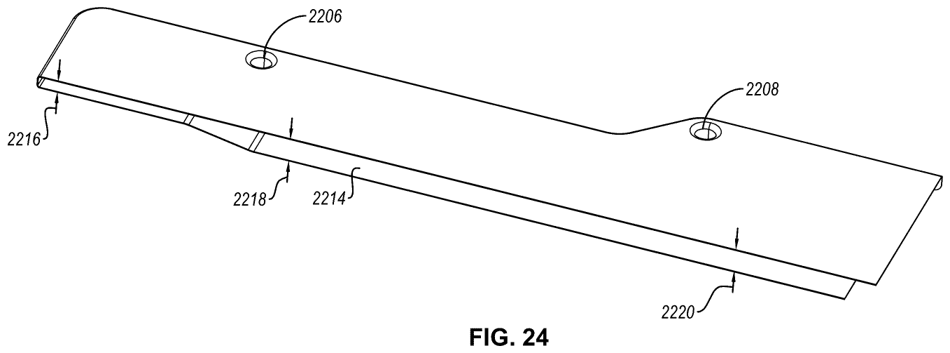

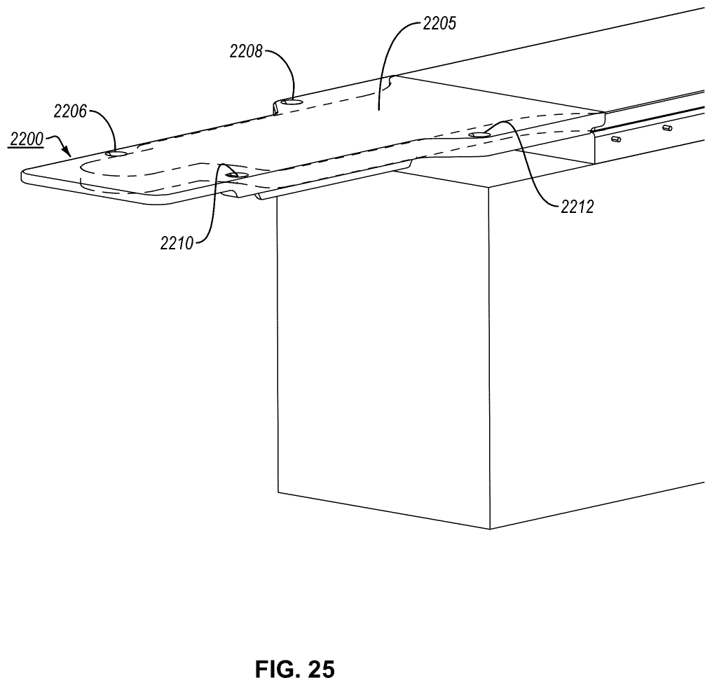

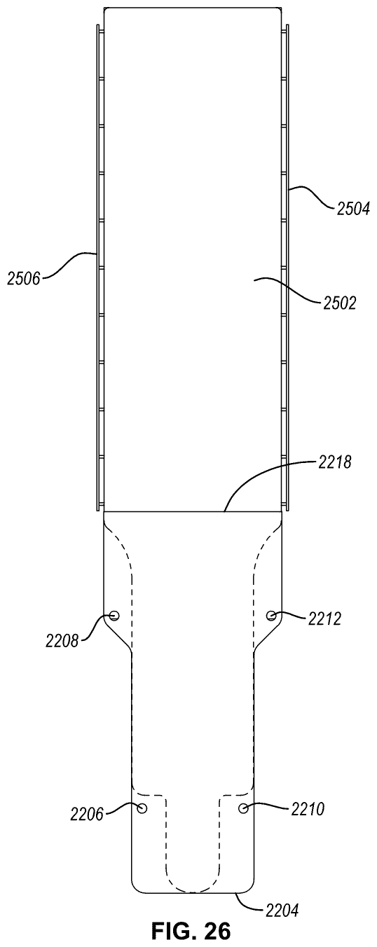

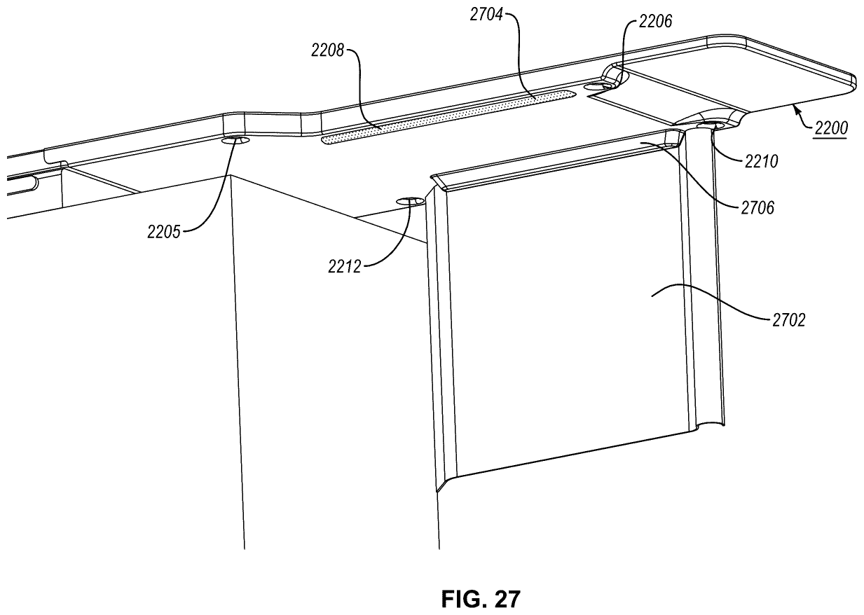

FIG. 22 illustrates a perspective rear end view of a table extension for a surgical table according to an embodiment of the invention. FIG. 23 illustrates a perspective rear end view of the table extension of FIG. 22 according to an embodiment of the invention. FIG. 24 illustrates a cross-section view of the table extension of FIG. 22 along line 2201 according to an embodiment of the invention. FIG. 25 illustrates a perspective side view of the table extension of FIG. 22 in an installed configuration according to an embodiment of the invention. FIG. 26 illustrates a top view of the table extension of FIG. 22 in an installed configuration according to an embodiment of the invention. FIG. 27 illustrates a top view of the table extension of FIG. 22 in an installed configuration according to an embodiment of the invention.

Referring to FIGS. 22-27, a surgical table extension is generally depicted with reference to number 2200. The surgical table extension 2200 includes one piece extension configured to be attached to an upper portion of a standard surgical table 2502. Optionally, and/or alternatively, the surgical table extension 2200 can be made with two pieces that join together.

In this embodiment, the surgical table extension 2200 is configured to increase the circumference and/or perimeter of a surgical table 2502. The oversizing of the surgical table extension 2200 as compared to at least portions of surgical table 2502 is further configured to permit one more radiation shields to better cover C-arm and mitigate scatter radiation by allowing the radiation shields to extend below the table in a wider manner by increasing the circumference or perimeter of the surgical table 2502.

The surgical table extension 2502 includes any attachment mechanism as described to releasably attach one or more surgical shields to the a perimeter of the surgical table extension 2200. In a preferred embodiment, the attachment mechanism includes a hook and loop attachment mechanism that corresponding to a hook and loop attachment mechanism on the radiation shield. Of course, other attachment mechanisms can be utilized, e.g., snaps, magnets, clips and combinations of the same and the like.

The surgical table extension 2200 includes a material that is substantially transparent to x-ray radiation or substantially a radiolucent material. In one embodiment, the surgical table extension 2200 includes one or a composite material, a carbon fiber material, a fiber-glass material, a plastic material, a thermoplastic material and combinations of the same and the like.

That surgical table extension 2200 includes a first end 2204 a spaced apart second end 2218, a first side 2207 and a second side 2209 spaced apart from the first side 2207.

The surgical table extension 2200 includes one or more attachment points, ports, recesses, holes, mechanisms and the like for receiving various attachment devices or components, e.g., radiation shielding components or adjustable mechanism that aid to support a portion of a body. In this embodiment, the surgical table extension 2200 include a first attachment unit 2206, a second attachment unit 2208, a third attachment unit 2210, and a fourth attachment unit 2212.

In this embodiment, the surgical table extension 2200 includes slot, recess or void 2214 extending substantially along a length of the surgical table extension 2200 that is configured to receive a portion the table 2502. The width of each of the slots 2216, 2218, and 2220, and can vary and size as creating an interference fit, e.g., a pressed fit or friction fit. Theses widths are sized to allow for fastening between an inner surface of the slot 2214 and an upper and lower surface of the surgical table 2502, thereby creating tightfitting mating parts that produces a joint which is held together by friction after the parts are pushed together. Optionally and/or alternatively, an adhesive, Velcro, pins, screws, pins, pegs, combinations of the same and the like can be used to further secure the surgical table extension 2202 to the surgical table 2502.

FIG. 25 illustrates a perspective side view of the table extension of FIG. 22 in an installed configuration according to an embodiment of the invention. FIG. 26 illustrates a top view of the table extension of FIG. 22 in an installed configuration according to an embodiment of the invention.

Referring to FIGS. 25-26, the surgical table extension 2200 is an installed configuration on the surgical table 2502. The surgical table 2502 also includes a first siderail 2504 and a second siderail 2506. In this embodiment, the surgical table extension 2200 is securely fastened with an interference fit as described herein.

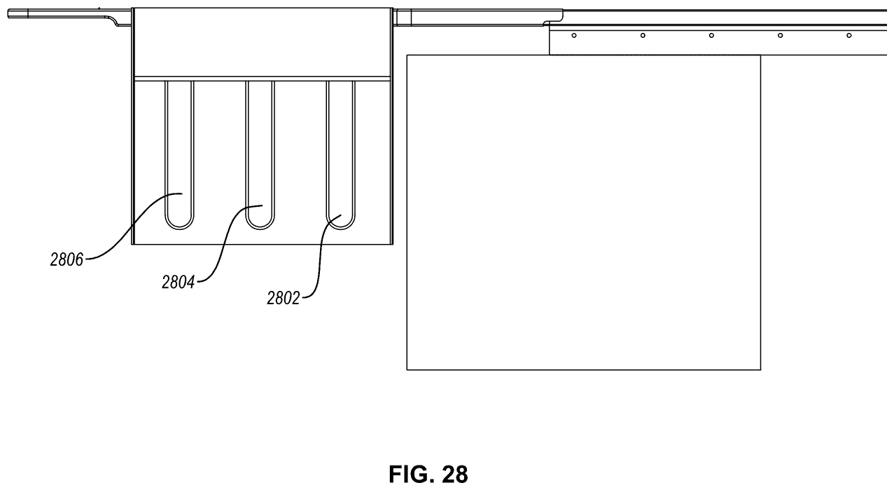

FIG. 27 illustrates a bottom side inside perspective view of the table extension of FIG. 22 with a flexible radiation shield installed configuration according to an embodiment of the invention. FIG. 28 illustrates a side perspective view of the table extension of FIG. 22 with a flexible radiation shield installed configuration according to an embodiment of the invention.

Referring to FIGS. 27-28, the surgical extension table 2202 is attached to a radiation shield 2702. In this embodiment, the radiation shield 2702 is described herein and can be adjusted vertically as described herein with one or more pneumatic channels of each adjustable segments 2802, 2804, and 2806. The surgical extension table 2202 includes a first bar 2704 and second bar 2706 configured with an attachment mechanism 2708 including a hook and loop mechanism or other attachment mechanism as described herein. The attachment mechanism 2708 is configured to be attached to an attachment mechanism on the radiation shield 2702. Of course, any type of attachment mechanism as described herein can be utilized.

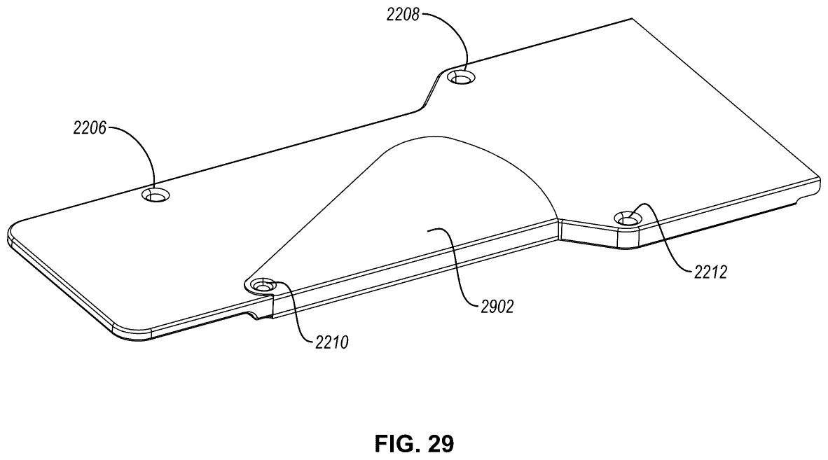

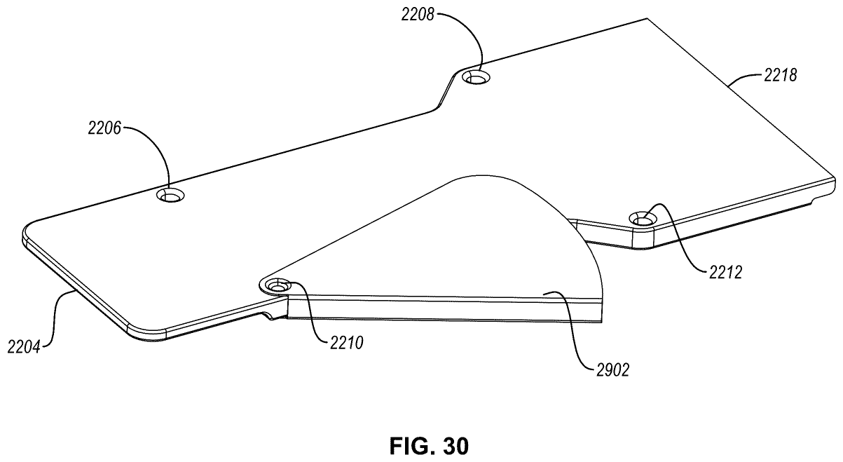

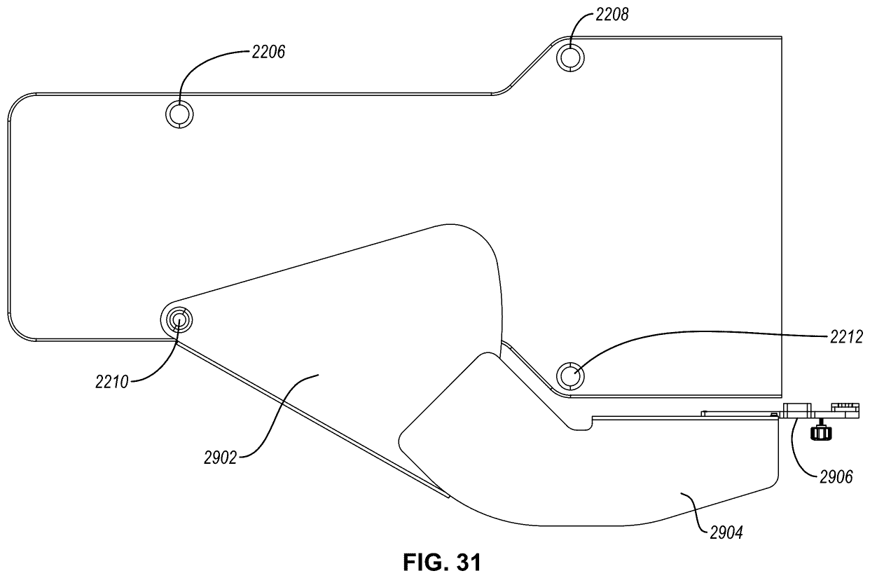

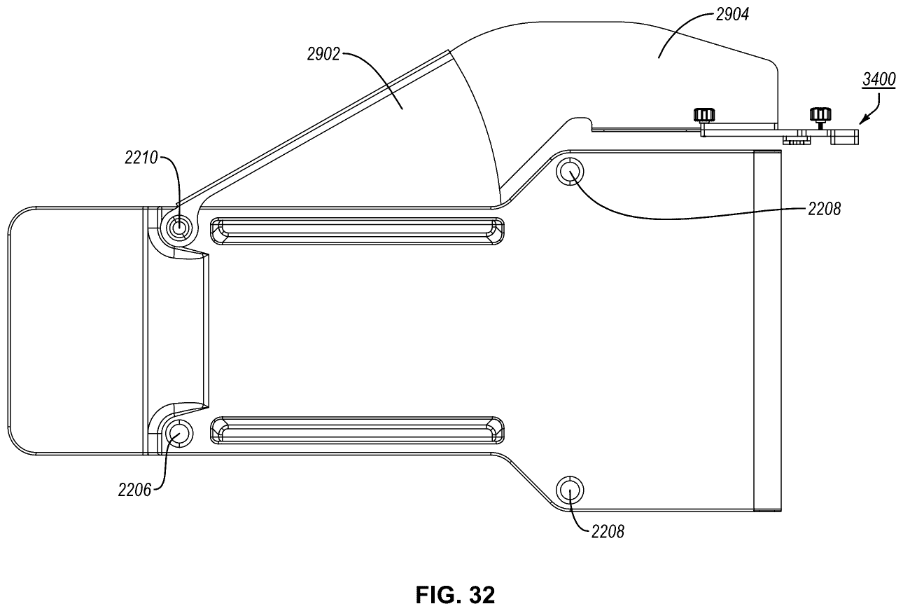

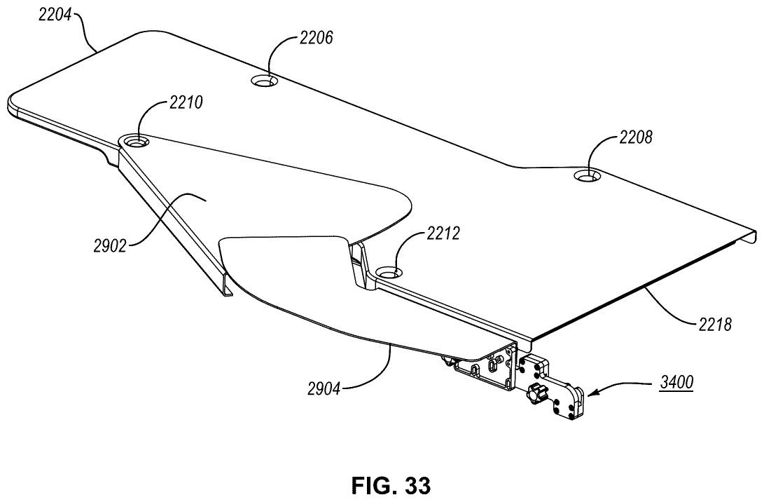

FIG. 29 illustrates a top perspective view of the table extension of FIG. 22 with an extension unit in a first orientation according to an embodiment of the invention. FIG. 30 illustrates a top perspective view of the table extension of FIG. 22 with an extension unit in a second orientation according to an embodiment of the invention. FIG. 30 illustrates a top perspective view of the table extension of FIG. 22 with an extension unit in a second orientation according to an embodiment of the invention. FIG. 31 illustrates a top view of the table extension of FIG. 22 with an extension unit and a bracket in a second orientation according to an embodiment of the invention. FIG. 32 illustrates a bottom view of the table extension, extension unit and bracket of FIG. 31. FIG. 33 illustrates a side perspective of the table extension, extension unit and bracket of FIG. 31.

Referring to FIGS. 29-33, the surgical table extension 2202 includes a first component 2902 rotionably attached to the attachment hole 2210. The first component or extension unit 2902 is configured to receive an arm or a patient and or can be used to receive surgical equipment. The first component 2902 is configured to rotate from about 0 degrees to about 90 degrees or greater relative an axis of the surgical table extension 2202.

Optionally, a second component or second extension unit 2904 is rotationably coupled to a first component 2902. The second component 2904 is configured to rotate from about 0 degrees to about 90 degrees or greater relative to a central axis of the surgical table extension 2202. In a preferred embodiment, the first extension unit 2902 and second extension unit 2904 is configured to rotate and receive at least a portion of an arm of a patient.

In this embodiment, the attachment hole 2210 includes a cylindrical reciprocal configured to receive a protrusion from the first extension unit 2902. The second extension unit 2904 is attached with a hinge. Optionally, the first extension unit 2902 and the second extension unit 2904 can be a one or two pieces and can be made of one or more radiation transparent material an composite materials, carbon fiber materials, fiberglass materials, plastic, thermoplastic, and combinations of the same. In this embodiment, a bracket 3400 is attached to the end portion of the second extension unit and configured to be attached to rail of the surgical table.

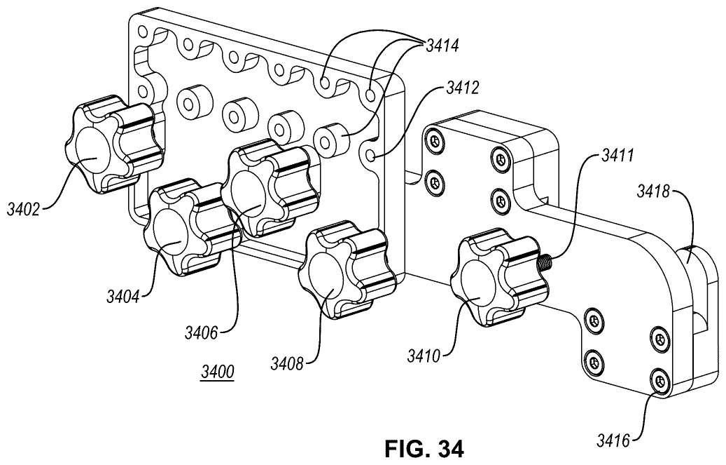

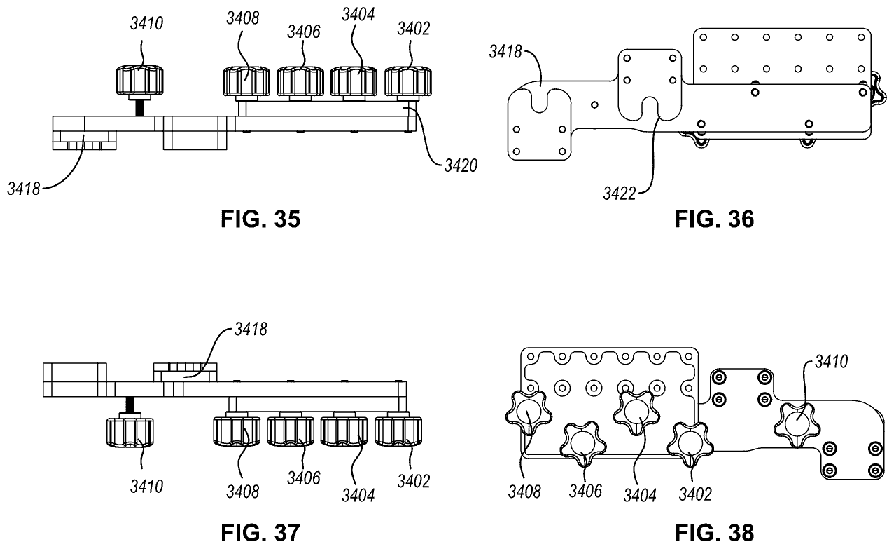

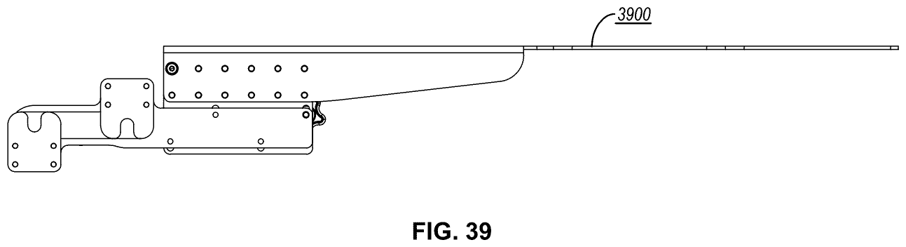

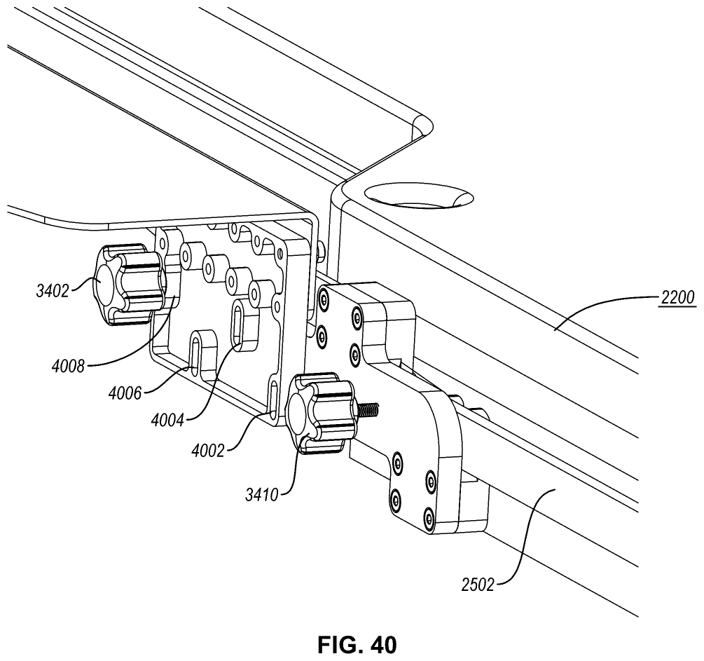

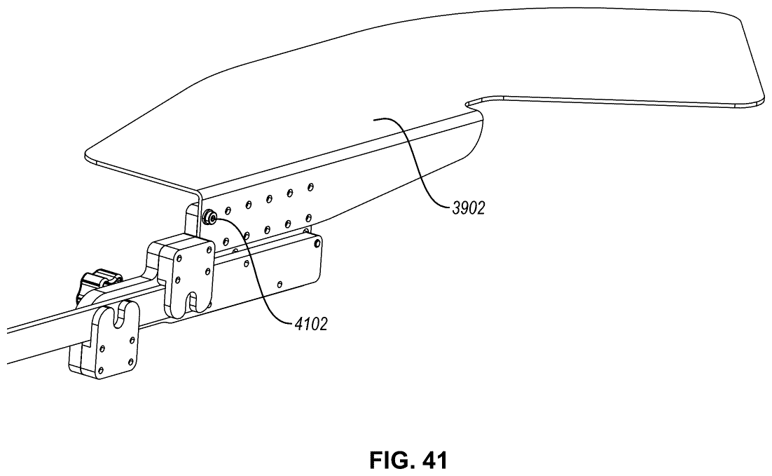

FIG. 34 illustrates a perspective right side view of a bracket according to an embodiment of the invention. FIG. 35 illustrates a top view of the bracket of FIG. 34 according to an embodiment of the invention. FIG. 36 illustrates a left side view of the bracket of FIG. 34 according to an embodiment of the invention. FIG. 37 illustrates a bottom view of the bracket of FIG. 34 according to an embodiment of the invention. FIG. 38 illustrates a right side view of the bracket of FIG. 34 according to an embodiment of the invention. FIG. 39 illustrates a left side view of the bracket of FIG. 34 with a table according to an embodiment of the invention. FIG. 40 illustrates a perspective view of a bracket of FIG. 34 coupled to a surgical table and attachment table. FIG. 41 illustrates a partial perspective view of a bracket of FIG. 34 coupled to a surgical table and attachment table.

Referring to FIGS. 34-41, a bracket is generally depicted with reference to number 3400. The bracket 3400 is configured to slidably and vertically engage a rail of a surgical table. That is, the bracket 3400 is configured to be vertically adjustable on a rail 2506 of surgical table 2502 and horizontal adjustable on the rail 2506 of the surgical table. In addition, the bracket is configured to receive one or more of table 3902, first extension unit 2902, second extension unit 2904, tray or other surgical apparatus. The vertical adjustment is configured to allow a medical personal to position the attachment flush (at the same level) with the surgical table, higher or lower as compared to the surgical table or surgical table extension 2202.

In this embodiment, the bracket 3400 includes an attachment knob 3402 positionable in vertical slot 4008, attachment knob 3404 is positionable in vertical slot 4006, attachment knob 3406 is positionable in vertical slot 4004, and attachment knob 3408 is positionable in vertical slot 4002. Each attachment knob 3402, 3404, 3406, and 3408 includes a thread and configured to engage the rail though their respective slots, 4008, 4006, 4004, and 4002, thereby allowing for vertical orientation of the bracket on the rail 2502. Of course, the bracket can contain fewer or more attachment knobs. The bracket includes a plurality of accessory ports, ports 3414 and 3412, configured to receive one or more of a table 3902, first extension unit 2902, second extension unit 2904, tray or other surgical apparatus. That is, the accessory ports allow for attachment with an attachment mechanism, e.g., screw, pin, peg, bolt, nut, combinations of the same and like.

The bracket 3400 also includes a knob 3410 with a thread 3411 configured to engage a rail 2502, thereby allowing for horizontal positioning of the bracket 3400. The bracket also includes stabilization slot 3418, stabilization slot 3420 and 3422 to engage the rail 2502 in a slidable manner. The slots 3418, 3420 and 3422 are a slightly oversized to receive at least a portion of the rails 2502. Referring to FIG. 41, the bracket 3400 is coupled to a table 3902. The table 3902 can be utilized by the medical personal.

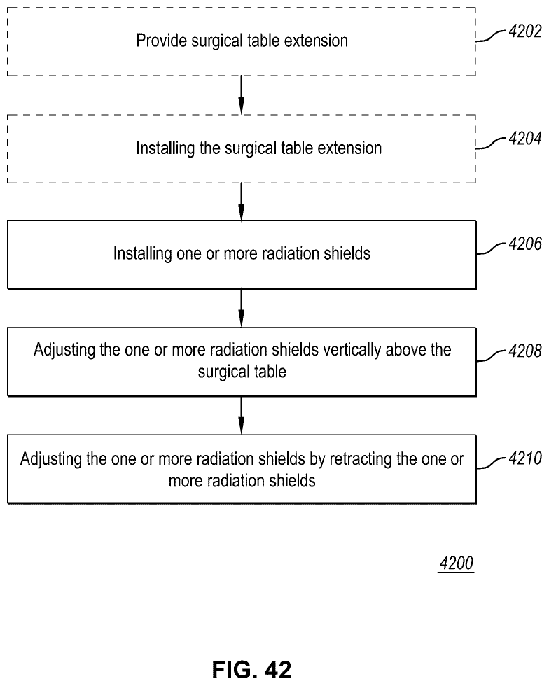

FIG. 42 illustrates a method of utilizing the extension table for the surgical table.

Referring to FIG. 42, the method 4200 includes providing the surgical extension (step 4202), arranging the surgical table extension on the surgical table and securing the surgical table extension on the surgical table (step 4204), steps 4202 and 4204 are optional. Installing one or more adjustable and/or inflatable radiation shields as described herein to the surgical table and/or the surgical table extension (step 4206), Next the radiation shield is adjusted in an up position (step 4208) with the control unit. Next the radiation shield is adjusted in a down position (step 4210) with the control unit.

It will be understood by those skilled in the art that numerous modifications, substitutions, and variations may be made to the embodiments described herein without departing from the spirit or scope of the invention. Accordingly, it is intended that the present disclosure encompass all such modifications and variations that fall within the scope of the appended claims and their legal equivalents.

The inventions, systems, devices, and methods described herein may be considered individually or collectively. Each embodiment may be practiced independently, or combined in whole or in part, in any suitable arrangement. Features and steps disclosed in connection with one embodiment may be incorporated into other embodiments as appropriate, and all such permutations and combinations are contemplated within the scope of this disclosure.

The subject matter described herein is intended to cover not only the specific embodiments explicitly disclosed, but also structural, functional, and procedural equivalents. Equivalent steps, elements, or processes, whether presently known or later developed, that perform substantially the same function in substantially the same way to achieve substantially the same result are intended to fall within the scope of the invention as defined by the appended claims.

The disclosure should also be understood as encompassing both broad and narrow aspects of the inventions. While certain embodiments are directed to specific applications or implementations, the underlying principles are not limited to such contexts and may be applied to other fields, industries, or uses. All such applications are considered within the scope of the claims.

To the extent that specific terms are used herein to describe embodiments, such terms are not intended to limit the scope of the invention but are used merely for clarity and convenience. The scope of the invention is defined solely by the appended claims, and all equivalents to the claims are expressly contemplated.

1. A system configured to reduce scatter radiation around a surgical table during a medical procedure, comprising:
at least one radiation shield configured to be connected to a surgical table with an attachment mechanism, wherein the at least one radiation shield includes one or more adjustable radiation shield segments coupled or releasably coupled to the at least one radiation shield;
a pump in fluid communication with the one or more adjustable radiation shield segments comprising a pneumatic channel that is configured to at least partially inflate the pneumatic channel such that at least a first portion of the at least one radiation shield extends vertically above at least a portion of the surgical table,
wherein a second portion of radiation shield is configured to extend below the surgical table; and
a controller configured to activate the pump.

2. The system of claim 1, wherein the at least one radiation shield comprises a first end having a first end portion, a second end having a second end portion spaced apart from the first end, wherein the pneumatic channel is sized to extend from a first end portion to the second end portion.

3. The system of claim 1, wherein the pneumatic channel further comprises one or more stiffing elements having each having a predetermined geometry, wherein the one or more stiffing elements comprises a region that is configured to bend an upper portion of the pneumatic channel at angle in range from about 1 degrees to about 35 degrees or greater relative to a vertical axis.

4.-5. (canceled)

6. The system of claim 1, when the pneumatic channel further comprises one or more stiffing elements configured to provide enhanced rigidity to the one or more adjustable radiation shield segments.

7. The system of claim 1, when the pneumatic channel further comprises one or more stiffing elements configured to bend at least an end portion of the at least one radiation shield that extends vertically above the surgical table at a predetermined angle relative to a vertical axis.

8. The system of claim 7, wherein the predetermined angle relative to the vertical axis is in a range from about 1 degrees to about 35 degrees.

9. The system of claim 7, wherein the end portion with the bend has a vertical length measured from a start of the bend to an end of the end portion in a range from about 1 inch to about 6 inches or greater.

10.-11. (canceled)

12. The system of claim 1, wherein the at least one radiation shield comprises a laminate structure comprising an outer layer, an inner layer and a middle layer arranged between the outer layer and the inner layer, wherein the middle layer comprises a radiation shielding material.

13.-16. (canceled)

17. The system of claim 12, wherein the radiation shielding material comprises a lead equivalence (LE) in a range from about 0.125 mm to about 1.0 mm or greater.

18.-22. (canceled)

23. The system of claim 1, wherein the pneumatic channel is configured to be inflated in a range from about 1 psi to about 50 psi or greater with the pump.

24. (canceled)

25. The system of claim 1, wherein the pneumatic channel is configured to be deflated with a vacuum pressure in a range from about 10⁻³ to 10⁻⁹ Torr with the pump.

26.-35. (canceled)

36. The system of claim 1, further comprising a control unit in communication with the controller, wherein the control unit is configured to control a manifold comprising one or more valves and the pump to individually extend or retract each of the plurality of radiation shields either independently, all at once or in any predetermined order.

37.-38. (canceled)

39. The system of claim 36, wherein the control unit is in communication over a network.

40. The system of claim 39, wherein the network comprises one or more of a wireless personal area network (WPAN), wireless local area network (WLAN), wireless metropolitan area network (WMAN), wireless wide area network (WWAN) and mobile networks.

41.-48. (canceled)

49. The system of claim 1, further comprising a surgical table extension, comprising:
a generally planar body having a top surface and a bottom surface; and
a recess in the generally planar body, the recess being open on one side and bounded on three sides by edge portions of the generally planar body including the top surface, the bottom surface and side surfaces extending from the top surface and the bottom surface, the recess is configured to receive at least a portion of the surgical table.

50. The system of claim 49, wherein the surgical table extension comprises a material that is substantially transparent to x-ray radiation.

51.-67. (canceled)

68. A system configured to reduce scatter radiation around a surgical table during
a medical procedure, comprising:
at least one flexible radiation shield comprising a first end, a second end spaced apart from the first end, a first side and a second side spaced apart from the first side, the at least one flexible radiation shield is configured to be attached to a surgical table with an attachment mechanism, wherein the at least one flexible radiation shield comprises one or more adjustable radiation shield segments comprising one or more air channels;
a pump in fluid communication with the one or more air channels, wherein the pump is configured to at least partially inflate the one or more air channels such that at least a first end of the at least one flexible radiation shield extends vertically above at least a portion of the surgical table, and
wherein a second end of flexible radiation shield is configured to extend below the surgical table; and
a controller in communication with a control unit, wherein the control unit is configured to activate the pump.

69. The system of claim 68, wherein the at least one flexible radiation shield comprises a laminate structure comprising an outer layer, an inner layer and a middle layer arranged between the outer layer and the inner layer, wherein the middle layer comprises a radiation shielding material.

70.-71. (canceled)

72. The system of claim 69, wherein the radiation shielding material comprises a material comprising a first material with a high atomic weight element and a second material with a lower atomic weight element.

73. A system configured to reduce scatter radiation around a surgical table during a medical procedure, comprising:
at least one flexible radiation shield comprising a first end, a second end spaced apart from the first end, a first side and a second side spaced apart from the first side, the at least one flexible radiation shield is configured to be attached to a surgical table with an attachment mechanism, wherein the at least one flexible radiation shield comprises one or more adjustable radiation shield segments comprising one or more air channels and one or more stiffing members;
a pump in fluid communication with the one or more air channels, wherein the pump is configured to at least partially inflate the one or more air channels such that at least a first end of the at least one flexible radiation shield extends vertically above at least a portion of the surgical table, and
wherein a second end of flexible radiation shield is configured to extend below the surgical table; and
a controller in communication with a control unit, wherein the control unit is configured to activate the pump.

74.-94. (canceled)
